(19) United States
(12) Patent Application Publication
(10) Pub. No.: US 2024/0156481 A1
(43) Pub. Date: May 16, 2024

(54) EXTERNAL WORK/SUCTION CHANNEL AND CLOT TRIMMER PASSING THERETHROUGH

(71) Applicant: ERGOGI LTD, Raanana (IL)

(72) Inventors: Gerard AGUILA, Zichron Yaakov (IL); Steven SHAMAH, Raanana (IL); Yassin ABU VASSEL, Raanana (IL)

(21) Appl. No.: 18/422,056

(22) Filed: Jan. 25, 2024

Related U.S. Application Data
(63) Continuation-in-part of application No. PCT/IL2022/050811, filed on Jul. 26, 2022.
(60) Provisional application No. 63/225,596, filed on Jul. 26, 2021.

(51) Int. Cl. A61B 17/32; A61B 17/00; A61B 90/00

(52) U.S. Cl. CPC A61B 17/32002; A61B 17/00234; A61B 90/39; A61B 2017/00017; A61B 2017/00296; A61B 2017/0034; A61B 2017/00982; A61B 2017/320032; A61B 2017/320052; A61B 2090/3937; A61B 2217/005

(57) ABSTRACT

A blood clot trimmer device and method for operating such is provided. The blood clot trimmer device is configured to pass through a work channel that is releasably attachable to an existing gastrointestinal examining device or through a working channel of an existing gastrointestinal examining device, and is configured to trim blood clots while the work channel that is releasably attachable to an existing gastrointestinal examining device is configured to remove trimmed blood clots from a site internal to a patient body via suction.

### TECHNICAL FIELD

The present disclosure relates to an external work channel that is configured to connect onto an existing endoscope, colonoscope, gastroscope or any gastroenterology device, and a clot trimmer configured to pass therethrough.

### BACKGROUND

Peptic ulcer disease can be caused by a variety of mechanisms that cause tissue ischemia to the surface of the gastric, duodenal or esophageal mucosa. Such tissue damage may be self-limited in case of erosive disease but may also cause ulceration in each of the aforementioned cavities or lumens. These ulcerations can cause pain and may further cause significant bleeding in the upper Gastrointestinal (GI) tract. Extensive bleeding may cause the gastric cavity to be filled with blood clots, which obscure the view of the cavity, thereby limiting proper evaluation during endoscopic examination.

Therefore, in case blood clots are present within the lumen of the upper GI tract, the blood clots need to be removed to allow proper visualization of the source of blood loss and therefore allow for proper treatment of the blood vessel. Current interventions for bleeding include angiography or surgery, but these are invasive and may be harmful to the patient necessitating the treatment. Other blood clot removal methods include use of suction and sometimes the addition of various blood clot breaking devices that occupy the existing working channel of the endoscope and are extremely cumbersome.

Similarly, blood clots may be present in the colon due to various conditions and diseases, such as diverticulosis, ischemic colitis, and colon cancer. In some cases, if hemostasis cannot be achieved, this may result in surgery to stop bleeding or invasive angiography, which is dangerous for the patient. Breaking up of blot clots may be performed by other devices in gastroenterology that occupy an existing working channel of a colonoscope, which are extremely cumbersome.

Accordingly, there is a need for a blood clot removal device that may be used during an endoscopic exam for upper GI bleed, or during a colonoscopy exam for colon bleed, that is easy to maneuver and operate, and does not occupy the existing working channel of the endoscope or colonoscope, such to enable operation of several devices in parallel.

### SUMMARY

As endoscopy and colonoscopy are the safest and most minimally invasive intervention for treating a patient suffering from gastrointestinal bleeding, a blot clot trimmer device that passes through an endoscope or colonoscope is hereby provided.

An aspect of an embodiment of the disclosure, relates to a blood clot trimmer device that is configured to pass through either a working channel of an existing endoscope/colonoscope (or any other GI device) or through an external work channel, which may be releasably attachable onto an existing endoscope/colonoscope through a cap device. In some embodiments, the external work channel may operate as an external suction channel. In some embodiments, the blood clot trimmer may pass through the external work channel or through the working channel of the endoscope/colonoscope, may be operated to trim a blood clot located along the gastrointestinal (GI) tract, and the trimmed blood clot may be removed through the external work channel, while the external work channel is used as an external suction channel, operated simultaneously with operation of the blood clot trimmer.

In some embodiments, the external work channel may comprise a distal endoscopic cap or colonoscopic cap, located at and attached to the distal end of the existing endoscope or colonoscope, respectively. When a blood clot is encountered, the external work/suction channel may be operated as an external suction channel, such to cause the blood clot to enter the distal cap. The blood clot trimmer, which may pass through the endoscope's or colonoscope's working channel, may then be activated while located within the endoscopic/colonoscopic cap, to trim that blood clot into smaller pieces, while suction is simultaneously applied, such that blood clot debris are continuously removed from the site internal to the patient's body through the external work/suction channel. In other embodiments, the external work/suction channel may be used both for passage of the blood clot trimmer as well as for suction used to remove fluid or tissue away from the patient's body.

An aspect of an embodiment of the disclosure, relates to a method for operating a blood clot trimmer device, which may include attaching an external work channel to an existing endoscope or colonoscope, passing the blood clot trimmer device through either the external work channel or the endoscope's/colonoscope's working channel, operating suction through the external work channel, trimming a blood clot using the blood clot trimmer device while simultaneously operating suction through the external work channel, and removing debris of the trimmed blood clot through the external work channel.

In some embodiments, a blood clot trimmer device is configured to pass through a work/suction channel that is releasably attachable to an existing endoscope/colonoscope or through a working channel of an existing endoscope/colonoscope is provided. The blood clot trimmer device is configured to trim blood clots and remove trimmed blood from a site internal to a patient bod. The blood clot trimmer device comprising: a flexible guide wire with a distal end and a proximal end; a sheath configured to enable free passage of the guide wire therethrough, the sheath including a distal end and a proximal end; a flexible tip connected to the distal end of the guide wire, the tip configured to rotate and thereby trim blood clots; a tip controller connected to the proximal end of the guide wire and to the proximal end of the sheath, the tip controller configured to control a degree of extraction or retraction of the tip with respect to the distal end of the sheath.

In some embodiments, the releasably attachable work/suction channel comprises an endoscopic cap or colonoscopic cap configured to releasably attach onto a distal end of an existing endoscope or colonoscope, respectively, the tip of the blood clot trimmer device configured to rotate within the endoscopic cap, whereby the releasably attachable work channel is configured to operate as a suction channel.

Optionally, the tip has a diamond shape. Optionally, the tip has a ‘duck-foot’ shape.

Optionally, the tip is made of nitinol.

Optionally, the tip controller comprises scale marks to indicate options for changing location of the tip with respect to the sheath.

Optionally, the tip controller comprises a tip location lock that is configured to move along the scale marks to determine the degree of extraction or retraction of the tip with respect to the distal end of the sheath, and to lock the determined position of the tip with respect to the distal end of the sheath.

Optionally, the blood clot trimmer device comprises a motor connected to the guide wire and a battery to provide power to the motor.

Optionally, movement of the motor causes movement of the guide wire and the tip connected to the guide wire with respect to the sheath, thereby determining degree or extraction or retraction of the tip with respect to the distal end of the sheath, further wherein operation of the motor is configured to rotate the tip.

Optionally, the blood clot trimmer device is manually operated via user rotation of a threaded handle rotating along a threaded rod.

Optionally, diameter of the releasably attachable work channel is between 3.7 mm to 4 mm.

Optionally, the blood clot trimmer device is configured to trim blood clots that the releasably attachable work channel is configured to remove via suction, to avoid aspiration of the trimmed blood clots.

Optionally, suction via the releasably attachable work channel is configured to operate simultaneously with operation of the blood clot trimmer device to thereby remove trimmed blood clots from the site during operation of the blood clot trimmer device.

Optionally, the releasably attachable work channel comprises ring shaped connectors to connect between the releasably attachable work channel and the existing endoscope/colonoscope, the ring-shaped connectors configured to enable passage of the existing endoscope/colonoscope therethrough.

In some embodiments, the blood clot trimmer device comprises a two-part handle comprising a reusable unit and a disposable unit. Optionally, the reusable unit comprises the motor and a power source, and the disposable unit comprises the flexible guide wire, the sheath, the flexible tip and the tip controller.

In some embodiments, the reusable unit comprises a protruding shaft, and the disposable unit comprises a corresponding groove configured to house the protruding shaft. Optionally, when the motor rotates, the shaft rotates, which in turn rotates the walls of the groove, which are connected to the guide wire, which is forced to rotate, thereby forcing the flexible tip to rotate and trim blood clots.

According to some embodiments, a method for operating a blood clot trimmer device that is configured to pass through a work channel that is releasably attachable to an existing endoscope/colonoscope or through a working channel of an existing endoscope/colonoscope is provided. The method comprising: providing a blood clot trimmer device comprising:
(i) a flexible guide wire with a distal end and a proximal end;
(ii) a sheath configured to enable free passage of the guide wire therethrough, the sheath including a proximal end and a distal end;
(iii) a flexible tip connected to the distal end of the guide wire, said tip configured to rotate and thereby trim blood clots; and
(iv) a tip controller connected to the proximal end of the guide wire and a proximal end of the sheath, the tip controller configured to control a degree of extraction or retraction of the tip with respect to the sheath.

In some embodiments, the releasably attachable work channel comprises an endoscopic/colonoscopic cap configured to releasably attach onto a distal end of an existing endoscope/colonoscope.

Optionally, the method comprises operating suction via the releasably attachable work channel to suck a blood clot from a site internal to a patient body into the endoscopic/colonoscopic cap.

Optionally, the method comprises operating rotation of the tip of the blood clot trimer device once a blood clot is sucked into the endoscopic/colonoscopic cap, thereby operating a trimming process of the sucked blood clot and breaking the sucked blood clot into pieces.

Optionally, the method comprises continuously operating suction by the releasably attachable work/suction channel to remove the pieces of the trimmed blood clot from the site, via the releasably attachable work channel, during the trimming process.

Optionally, the method comprises moving a tip location lock along scale marks on the tip controller, thereby determining the degree of extraction or retraction of the tip with respect to the distal end of the sheath.

Optionally, the method comprises locking the determined position of the tip with respect to the distal end of the sheath to prevent further movement of the tip with respect to the sheath.

Optionally, the blood clot trimmer device comprises a motor, such that the method further comprises causing movement of the guide wire and the tip connected to the guide wire with respect to the sheath by moving the motor, thereby determining degree or extraction or retraction of the tip with respect to the distal end of the sheath, and operating the motor for rotating the tip.

### BRIEF DESCRIPTION OF THE DRAWINGS

The present disclosure will be understood and better appreciated from the following detailed description taken in conjunction with the drawings. Identical structures, elements or parts, which appear in more than one figure, are generally labeled with the same or similar number in all the figures in which they appear, wherein:

FIG. 1 is a schematic illustration of an external suction and work channel device, according to an embodiment of the disclosure;

FIG. 2 is a schematic illustration of an external suction and work channel device attached to an endoscope, according to an embodiment of the disclosure;

FIG. 3 is a schematic illustration of a blood clot trimmer device, according to an embodiment of the disclosure;

FIGS. 4A-4B are schematic illustrations of a blood clot trimmer device tip when the blood clot trimmer blades are inside the sheath, and when the blades are outside the sheath, respectively, according to an embodiment of the disclosure;

FIG. 5A is a schematic illustration of a side view of a tip of a blood clot trimmer device with its blades extracted outside of the sheath and possibly outside of the work channel, according to an embodiment of the disclosure;

FIG. 5B is a schematic illustration of a front view of the blades of the blood clot trimmer device when extracted outside the work channel, according to an embodiment of the disclosure;

FIG. 5C is a schematic illustration of a front view of the blades of the blood clot trimmer device when extracted outside of the sheath while retracted into the work channel, according to an embodiment of the disclosure;

FIG. 6 is a schematic illustration of the blood clot trimmer device passing through the external suction and work channel device, according to an embodiment of the disclosure;

FIG. 7 is a schematic illustration of a physician maneuvering a blood clot trimmer device and an external work channel device, according to an embodiment of the disclosure;

FIG. 8 is a schematic illustration of two options through which a blood clot trimmer device can pass, one is through an external suction and work channel device and a second is through an existing work channel of an endoscope, according to embodiments of the disclosure;

FIG. 9 is a flow chart illustrating a method for operating the blood clot trimmer device, according to an embodiment of the disclosure;

FIG. 10 is a schematic illustration of an external suction and work channel device, according to another embodiment of the disclosure;

FIG. 11 is a schematic illustration the an external suction and work channel device attached to an endoscope, according to an embodiment of the disclosure;

FIG. 12 is a schematic illustration of a blood clot trimmer device, according to another embodiment of the disclosure;

FIGS. 13A-13B are schematic illustrations of a blood clot trimmer device tip when the blood clot trimmer tip is inside and outside the sheath, and when the tip is outside the sheath but inside the distal cap, respectively, according to an embodiment of the disclosure;

FIG. 14A-14C are schematic illustrations of the tip of the blood clot trimmer device inside the distal cap, in cross-section view, front-perspective view and side-perspective view during rotation of the tip, respectively, according to an embodiment of the disclosure;

FIG. 15 is a schematic illustration of two options through which a blood clot trimmer device can pass, one is through an external suction and work channel device and a second is through an existing work channel of an endoscope, according to embodiments of the disclosure;

FIG. 16 is a schematic illustration of the blood clot trimmer device passing through the external suction and work channel device, according to an embodiment of the disclosure;

FIG. 17 is a schematic illustration of the blood clot trimmer device passing through an existing work channel of an endoscope, according to an embodiment of the disclosure;

FIG. 18 is a schematic perspective view of the blood clot trimmer device, from handle to tip, according to an embodiment of the disclosure;

FIGS. 19A-19B are schematic illustrations of the blood clot trimmer device, in side view and cross-section view, respectively, according to an embodiment of the disclosure;

FIGS. 20A-20C are schematic illustrations of a motorized blood clot trimmer device in side view, cross-section view when the trimmer tip is retracted from the endoscopic cap, and a cross-section view when the trimmer tip is extracted and positioned within the endoscopic cap, respectively, according to embodiments of the disclosure;

FIGS. 21-21D are schematic illustrations of a manual blood clot trimmer device, and enlarged views of the manual blood clot trimmer's circular connection, linear to radial movement, and the guide wire retraction mechanism, respectively, according to an embodiment of the disclosure;

FIGS. 22A-22B are schematic illustrations of a multi-lumen accessory suction and work channel, according to embodiments of the disclosure;

FIG. 23 is a flow chart illustrating a method for operating the blood clot trimmer device, according to an embodiment of the disclosure;

FIGS. 24A-24D are schematic illustrations of a side-perspective view of the two-part handle of the blood clot trimmer device, a side-perspective view of the disposable unit of the handle, a rear-perspective view of the disposable unit of the handle, and a side-perspective view of the reusable unit of the handle, according to embodiments of the present disclosure;

FIGS. 25A-25B are schematic illustrations of a cross-section of the reusable unit, and of the two-part handle of the blood clot trimmer device, respectively, according to embodiments of the present disclosure;

FIGS. 26A-26B are schematic illustrations of an enlarged side-perspective view and a side-perspective view, respectively, of a blood clot trimmer, according to embodiments of the present disclosure; and

FIGS. 27A-27B are schematic illustrations of cross-sections of a blood clot trimmer device tip when the blood clot trimmer tip is outside the sheath but inside the distal cap, and of a clot trimmer tip alone, respectively, according to embodiments of the present disclosure.

### DETAILED DESCRIPTION

In order to remove blood clots from a site internal to a patient body, e.g., the gastrointestinal tract, or to remove blood clots that may be resultant of a gastrointestinal tract ulcer, which may obscure the visualization of a hemorrhaging blood vessel during gastrointestinal intervention, while avoiding systemic or invasive solutions, a blood clot trimmer device may be operated via an external work/suction channel of an endoscope, colonoscope or any other GI tract examining device.

According to the present disclosure, the term “endoscope” may be interchangeable with the terms “colonoscope”, “gastroscope”, or any other gastrointestinal tract examining device, which includes gastrointestinal imaging devices.

According to the present disclosure, the term “endoscopic”, may be interchangeable with “colonoscopic”, “gastroscopic”, or with any other relationship to any other gastrointestinal tract examining device, which includes gastrointestinal imaging devices.

The external work channel may be connected to an existing endoscope by use of an endoscopic cap attached to the distal end of the endoscope, and the blood clot trimmer device may be configured to pass through the external suction and work channel. In some embodiments, other devices besides the blood clot trimmer device may be passed through the external work channel to perform other procedures while keeping the existing endoscope's work channel free for yet other or additional tools and therapeutics.

In yet other embodiments, the blood clot trimmer device may be passed through the existing work channel, while the external work channel may be used for suction.

In some embodiments, suction may be operated before operation of the blood clot trimmer device, e.g., to suck a blood clot into the distal endoscopic cap, and may further be operated during operation of the blood clot trimmer device that breaks or cuts the blood clot into pieces, such to suck the debris or the pieces of the broken blood clot out of the internal site of the patient's body, where the blood clots were present.

The terms “external work channel”, “external work and suction channel”, “external suction channel”, “external suction and work channel”, “accessory work channel”, “accessory work and suction channel”, “accessory suction and work channel”, “accessory suction channel”, and “accessory work/suction channel”, or any combination thereof, are interchangeable throughout the description, and refer to a work and/or suction channel that are in addition to existing work and suction channels that are an inherent part of any endoscope.

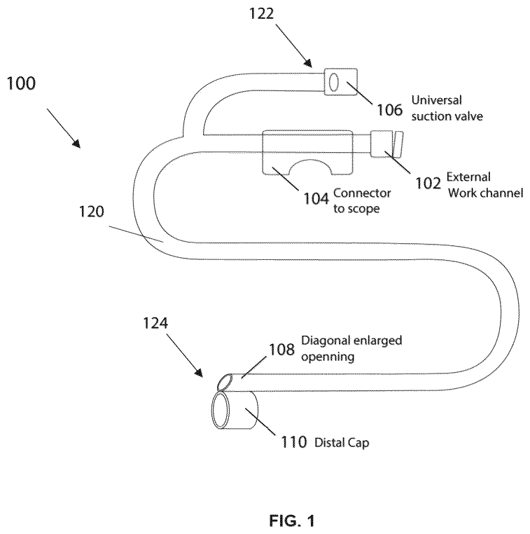

FIG. 1 is a schematic illustration of an external work channel device, according to an embodiment of the disclosure. In some embodiments, external work channel device 100 may comprise an external work channel cap 102 through which an operating device, e.g., a blood clot trimmer device, may enter the external work channel device 100. In some embodiments, external work channel device 100 may comprise a connector 104, which connects the external work channel device 100 to a scope handle of an existing endoscope.

In some embodiments, external work channel device 100 may further comprise a suction and work channel 120, through which the device that is to operate inside the gastrointestinal tract, is passed after entering through external work channel cap 102. In some embodiments, the proximal end 122 of suction and work channel 120 may be connected to external work channel cap 102, while the distal end 124 of suction and work channel 120 may be cut such to create a diagonal opening 108, which provides an enlarged opening compared to an opening straightly cut. In some embodiments, external work channel device 100 may further comprise a cap 110, configured to attach the distal end of suction and work channel 120 to the distal end of an endoscope to which external suction and work channel device 100 is connected. Cap 110 may assist in stabilizing the distal end of external work channel device 100 by connecting it to the distal end of the endoscope. Furthermore, cap 110 may enable a user, e.g., a physician, operating the endoscope to easily view the area of operation of the operating device passing through the external suction and work channel device 100 via the scope and optics located at the distal end of the endoscope.

In some embodiments, external suction and work channel device 100 may further comprise a suction valve 106, which may be any universal off-the-shelf suction valve available. A suction valve such as suction valve 106 may be in use when either fluid or tissue should be removed from the upper GI tract environment, e.g., to enhance better vision of the operating area. In some embodiments, suction valve 106 may be used to enable removal of a trimmed blood clot from inside the upper GI tract.

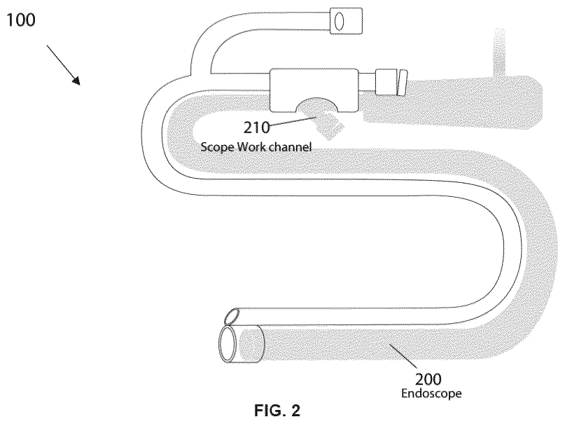

FIG. 2 is a schematic illustration of an external suction and work channel device attached to an endoscope, according to an embodiment of the disclosure. In some embodiments, external suction and work channel device 100 may be releasably attachable to an existing endoscope. In some embodiments, external suction and work channel device 100 may be connected via connector 104 to an existing endoscope, e.g., endoscope 200, which may comprise its own work channel 210. In some embodiments, cap 110 may also connect the distal end 124 of external suction and work channel device 100 to the distal end of the endoscope, e.g., endoscope 200.

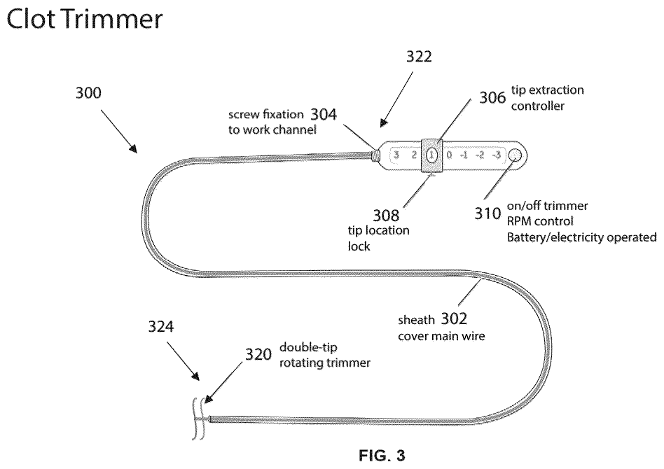

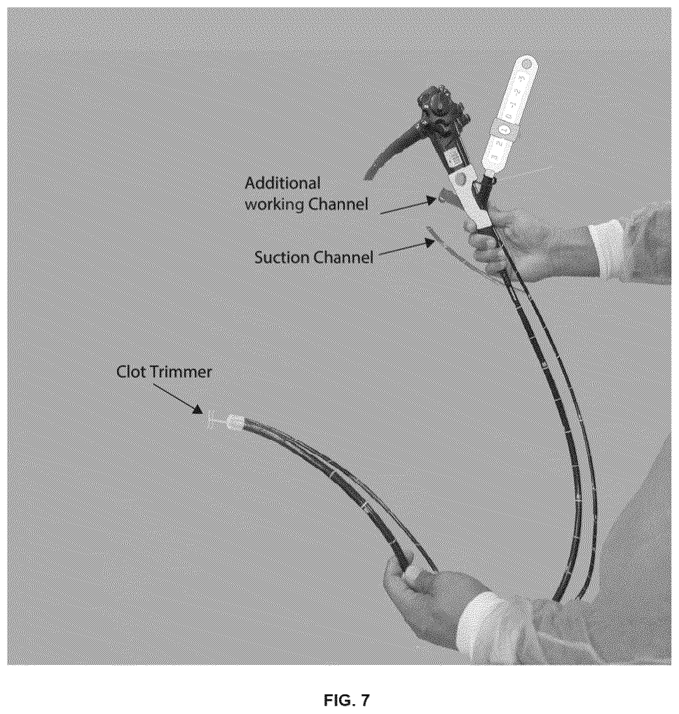

FIG. 3 is a schematic illustration of a blood clot trimmer device, according to an embodiment of the disclosure. In some embodiments, a blood clot trimmer device 300 is provided. Optionally, blood clot trimmer device 300 is configured to pass through an external work channel device, e.g., work channel device 100, as illustrated in FIG. 6.

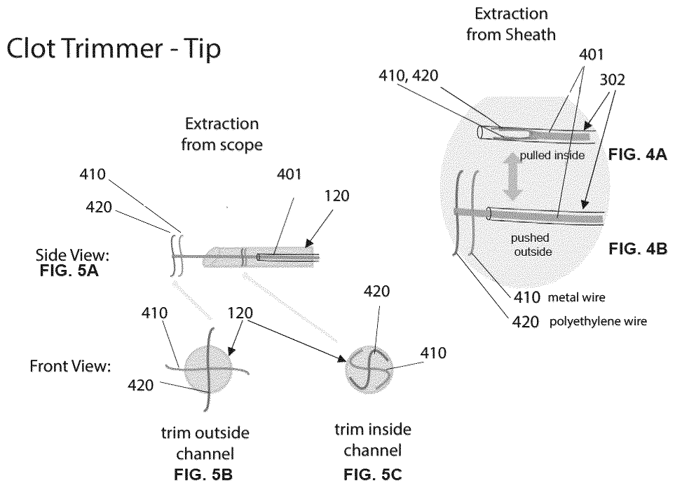

According to some embodiments, blood clot trimmer device 300 may comprise a sheath 302 through which a clot trimer tip 320 may exit at the distal end 324 of sheath 302. Optionally, clot trimmer tip 320 may comprise at least two wires acting as blades configured to cut blood clots. As illustrated in FIG. 4B, the at least two wires of tip 320 may comprise a rigid wire 410, which may be made, for example, of metal, and a flexible wire 420, which may be made, for example, of flexible plastic, such as polyethylene. The rigid wire 410 may be typically located behind the flexible wire 420 at a certain distance. In some embodiments, at least two wires 410 and 420 may rotate and thus trim or cut blood clots during their rotation.

In some embodiments, blood clot trimmer device 300 may further comprise a tip extraction controller 306, which may control the extraction and/or the retraction of clot trimer tip 320 outside and/or inside of the distal end 324 of sheath 302. Optionally, tip extraction controller 306 may comprise a dial or a bar of numbers, which may indicate different possible distances of clot trimer tip 320 either outside of distal end 124 of suction and work channel 120 or inside distal end 124 of suction and work channel 120. In some embodiments, tip extraction controller 306 may comprise a tip location lock 308, which a user may move along the numbers representing distances on tip extraction controller 306, to lock a specific distance to which the clot trimmer tip 320 may be pushed (extracted) or pulled (retracted) out and in of suction and work channel 120.

For example, in case tip extraction controller 306 comprises several positive numbers, a zero and several negative numbers, the positive numbers may indicate the degree of extraction of clot trimer tip 320 outside of suction and work channel 120, the zero may indicate that clot trimer tip 320 is located at the edge of distal end 124 of suction and work channel 120, and the negative numbers may indicate the degree of retraction of clot trimer tip 320 inside of suction and work channel 120. Other markings may be used to indicate retraction and extraction of clot trimer tip 320 in and out of suction and work channel 120.

In some embodiments, sheath 302 may comprise fixation means 304, e.g., a screw, on its proximal end 322, to fixate sheath 302 to the scope channel of the endoscope along which blood clot trimmer device 300 is inserted into a site internal to a patient body, e.g., the gastrointestinal tract, via an external work channel device, for example, external work channel device 100.

In some embodiments, blood clot trimmer device 300 may further comprise an on/off operation mechanism 310, e.g., a press button, which may initiate or cease operation of blood clot trimmer device 300, and may further control revolutions per minute or speed of rotation of the at least two wires at tip 320. Optionally, blood clot trimmer device 300 may comprise a power supply (not shown), e.g., a battery, a rechargeable battery or an electricity connection, which may be operated by pressing button 310. When the power supply is operated, it causes the trimming wires 410 and 420 to rotate and thus enable trimming and cutting of blood clots.

FIGS. 4A-4B are schematic illustrations of a blood clot trimmer device tip 320 when the blood clot trimmer blades are inside the sheath, and when the blades are outside the sheath, respectively, according to an embodiment of the disclosure. FIG. 4A illustrates wires or blades 410 and 420 that are connected to a guide 401 when retracted into of sheath 302. In some embodiments, sheath 302 is configured to cover guide 401. That is, as illustrated in FIG. 4A, guide 401 is pulled back into sheath 302, e.g., by locking tip location lock 308 on tip extraction controller 306 on a number representing a retraction position. On the contrary, FIG. 4B illustrates guide 401 pushed outside sheath 302, which causes wires or blades 410 and 420 to be extracted outside of sheath 302, for example, to engage a trimming process.

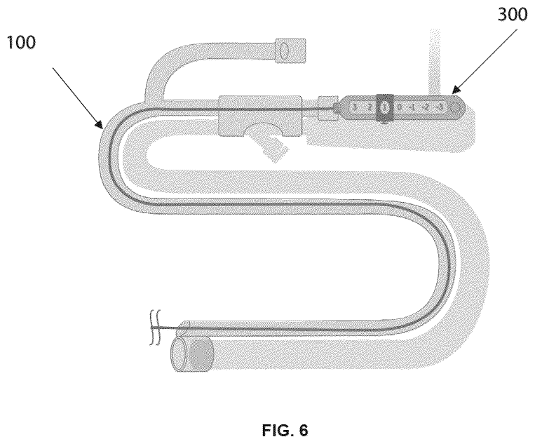

FIG. 5A is a schematic illustration of a side view of a tip of a blood clot trimmer device with its blades extracted outside of the sheath and optionally outside of suction and work channel 120, according to an embodiment of the disclosure. In some embodiments, when blood clot trimmer device 300 is passed along a suction and work channel of external work channel device 100, e.g., suction and work channel 120, blood clot trimmer device tip 320 may not only be pushed outside of sheath 302 but may be extracted outside of suction and work channel 120 altogether.

FIG. 5A illustrates two options—a blood clot trimmer tip 320 when extracted from sheath 302 but still located inside suction and work channel 120, as further illustrated in FIG. 5C, and a blood clot trimmer tip 320 when extracted from both the sheath 302 and suction and work channel 120, as further illustrated in FIG. 5B.

As illustrated in FIG. 5A with respect to the second option (FIG. 5B), guide 401 is pushed outside of suction and work channel 120, such that blood clot trimmer device tip 320 comprising at least rigid wire 410 and flexible wire 420 is extracted outside of suction and work channel 120.

FIG. 5B is a schematic illustration of a front view of the blades of the blood clot trimmer device when extracted outside the sheath and further outside suction and work channel 120, according to an embodiment of the disclosure. When at least two blades 410 and 420 are extracted outside of suction and work channel 120, these blades may be rotated by a motor (not shown), for example, when the power supply that is configured to supply power to the motor is initiated. Blades 410 and 420 may be positioned substantially perpendicularly to one another, such to be located at an angle of approximately 90 degrees between one blade to another, while flexible blade 420 is located in front of and at a certain distance from rigid blade 410. In other embodiments, at least two blades 410 and 420 may be positioned at different locations per one another.

When rotated, flexible blade 420 that is located at the front end of blood clot trimmer device tip 320 may rotate to trim and cut a blood clot present in the gastrointestinal tract, without causing any harm to surrounding tissue, due to its flexibility characteristics. Rigid blade 410 may enable complete trimming when brought closer to the surface of the tissue.

In some embodiments, the speed of rotation of each of the at least two blades 410 and 420 may be controlled separately. That is, a user may choose to operate flexible blade 420 at a lower speed compared to the speed of rigid blade 410, or vice versa. This may be implemented by designing a separate sheath or guide per each of the at least two blades 410 and 420 and connecting each to a motor and power supply.

FIG. 5C is a schematic illustration of a front view of the blades of the blood clot trimmer device when retracted inside the work channel while being extracted outside of sheath 302, according to an embodiment of the disclosure. In some embodiments, when retracted, e.g., pulled into suction and work channel 120, since each of the at least two blades 410 and 420 is longer than the diameter of suction and work channel 120, each of the at least two blades 410 and 420 is folded within the diameter of suction and work channel 120. When folded, each of the at least two blades 410 and 420 may create an ‘S’ shape, while maintaining a substantially perpendicular location of one blade with respect to the other.

In some embodiments, even when blood clot trimmer device tip 320 is located inside suction and work channel 120, e.g., at the edge of the distal end 124 of suction and work channel 120, after tip 320 is extracted from sheath 302, the at least two blades 410 and 420 may be operated such to rotate and trim a blood clot, when blood clot trimmer device tip 320 is pressed against the blood clot's surface or edges. In some cases, such trimming is a more focused trimming, which may cause less harm to tissue surrounding the blood clot.

FIG. 6 is a schematic illustration of the blood clot trimmer device passing through the external work channel device, according to an embodiment of the disclosure, and FIG. 7 is a schematic illustration of a physician maneuvering a blood clot trimmer device and an external work channel device, according to an embodiment of the disclosure. According to some embodiments, a blood clot trimmer device, e.g., blood clot trimmer device 300 may pass through an external work channel device, e.g., external work channel device 100, in order to provide blood clot trimming capabilities without occupying an existing work channel of an existing endoscope. As explained with respect to FIG. 1, external work channel device 100 may be connected to an endoscope via a connector 104 and possibly via cap 110. And as explained with respect to FIG. 3, and as illustrated in FIG. 6, blood clot trimmer device 300 may pass through an external work channel device, while possibly being connected to the work channel of the endoscope via fixation means 304.

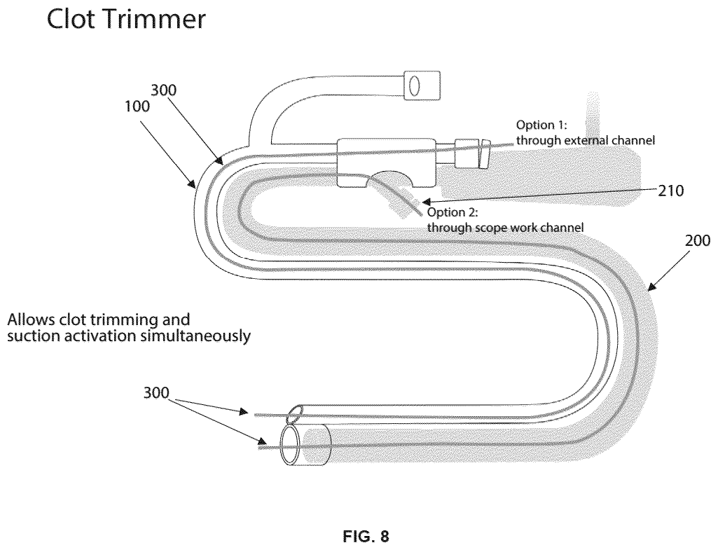

FIG. 8 is a schematic illustration of two options through which a blood clot trimmer device can pass, one is through an external suction and work channel device and a second is through an existing work channel of an endoscope, according to embodiments of the disclosure. In some embodiments, blood clot trimmer device 300 may be passed through an external suction and work channel device, e.g., through external suction and work channel device 100. Whereas, in other embodiments, blood clot trimmer device 300 may be passed through an existing work channel or an existing endoscope, e.g., through work channel 210 of endoscope 200.

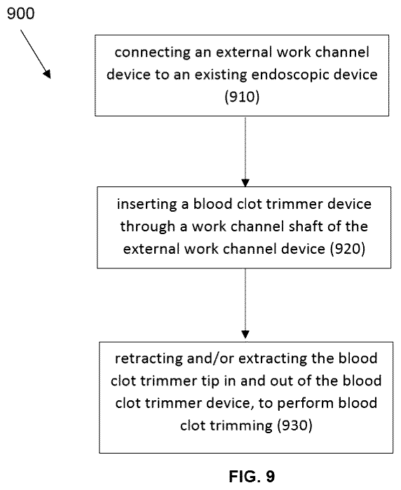

FIG. 9 is a flow chart illustrating a method for operating the blood clot trimmer device, according to an embodiment of the disclosure. In some embodiments, a method 900 for operating a blood clot trimmer device, e.g., blood clot trimmer device 300, may optionally comprise operation 910, which may comprise connecting an external work channel device to an existing endoscope. In some embodiments, method 900 may further comprise operation 920, which may comprise inserting a blood clot trimmer device through a work channel of the external work channel device. However, in other embodiments, a blood clot trimmer device, e.g., blood clot trimmer device 300 may be passed through the existing work channel of an existing endoscope, without the need to first connect an external work channel to the endoscope, as illustrated in FIG. 8.

In some embodiments, method 900 may comprise operation 930, which may comprise retracting and/or extracting the blood clot trimmer tip in and out of the blood clot trimmer device, to perform blood clot trimming That is, according to some embodiments, a user may determine the amount of extraction or retraction of the rotating trimming tip of blood clot trimmer device, e.g., clot trimer tip 320, according to the type of blood clot and the preferred way of operating the blood clot trimmer device. For example, in some cases, a user may prefer to extract the rotating tip of the blood clot trimmer device, in order to remove a blood clot, possibly a blood clot that is substantially protruding from the surface of a blood vessel along the gastrointestinal tract.

Whereas in other cases, a user may prefer to extract clot trimmer tip 320 out of the distal tip of sheath 302 but to retract the rotating tip 320 of the blood clot trimmer device up to the edge of the work channel, whether the work channel is an external work channel such as suction and work channel 120, or whether it is an existing work channel of the endoscope. For example, rotating trimer tip 320 may be extracted from sheath 302 and be retracted up to the edge of the distal end 124 of the external work channel, e.g., suction and work channel 120, in order to remove a blood clot that is less protruding above the surface of a blood vessel along the intestine.

In some embodiments, the trimmed blood clot may be removed through the external work channel device, once the blood clot trimmer device is retracted through the external work channel device.

In some embodiments, the suction process, e.g., initiated via suction valve 106, may be operated independently or simultaneously to the blood clot trimming operation.

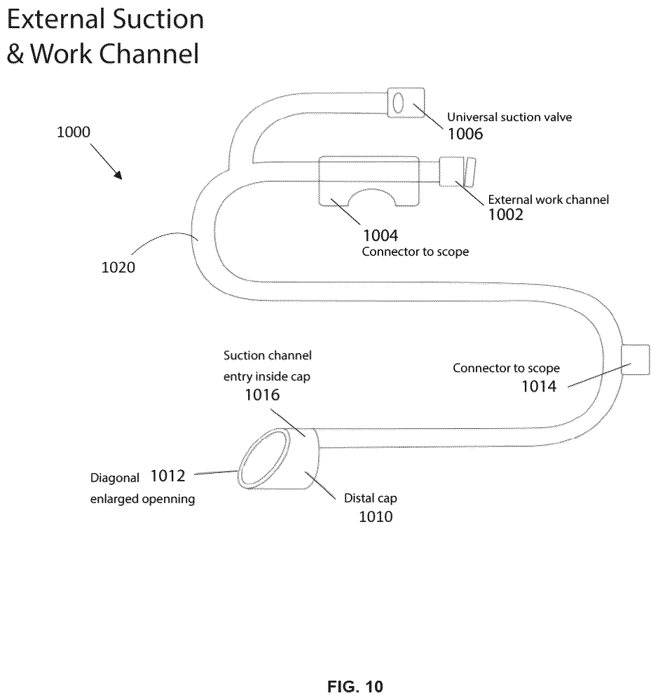

FIG. 10 is a schematic illustration of an external/accessory work channel device, according to an embodiment of the disclosure. In some embodiments, accessory work/suction channel device 1000 may comprise an accessory work channel opening/cap 1002 through which an operating device, e.g., a blood clot trimmer device, may enter the external work channel device 1000. In some embodiments, accessory work channel device 1000 may comprise a connector 1004, which connects the external work channel device 1000 to a scope handle of an existing endoscope.

In some embodiments, external suction and work channel device 1000 may further comprise a suction valve 1006, which may be any universal off-the-shelf suction valve available. A suction valve such as suction valve 1006 may be in use when either fluid or tissue should be removed from the patient's body, e.g., the intestine environment, to enhance better vision of the operating area. In some embodiments, suction valve 1006 may be used to enable removal of a trimmed blood clot from inside the GI tract.

In some embodiments, accessory work channel device 1000 may further comprise an accessory suction and work channel 1020, which is bifurcated to the work channel opening 1002 and to suction valve 1006. That is, accessory work/suction channel 1020 may be used for passage of an instrument therethrough, and/or for operating suction (i.e., vacuum) therethrough. In some embodiments, a device that is to operate inside an internal site of the patient's body, e.g., the patient's gastrointestinal tract, is passed through accessory work/suction channel 1020 after entering through external work channel cap 1002. In some embodiments, accessory suction and work channel 1020 may have a diameter of between 3.7 mm to 4 mm, which is significantly larger than the diameter of a standard suction channel added to an existing endoscope, and further larger than the diameter of an existing work channel of endoscopes. Thus, the accessory suction and work channel 1020 of the present disclosure is able to remove larger pieces of debris from an internal site of the patient's body compared to current endoscopes. For example, accessory suction and work channel 1020 of the present disclosure is able to remove large pieces of blood clots that are cut by a blood clot trimmer of the present disclosure, as will be described later with respect to blood clot trimmer 3000. In some embodiments, accessory work/suction channel 1020 may be made of Thermoplastic Polyurethane, though other materials may be used.

In some embodiments, the proximal end of suction and work channel 1020 may be created by merging a channel beginning at suction valve 1006 and a channel beginning at external work channel cap 1002. In some embodiments, the distal end of suction and work channel 1020 may comprise a distal endoscopic cap 1010, which may be cut such to create a diagonal opening 1012, which provides an enlarged opening compared to an opening straightly cut, and thus may further provide easier intubation of the esophagus. In some embodiments, an extended field of view is provided, due to endoscopic cap 1010, which is typically transparent. Endoscopic cap 1010 may be made of an elastomer, e.g., Thermoplastic Polyurethane, though other materials may be used.

In some embodiments, the distal endoscopic cap 1010 may be configured to attach the distal end of suction and work channel 1020 to the distal end of an endoscope to which external suction and work channel device 1000 is connected. Endoscopic cap 1010 may assist in stabilizing the distal end of external work channel device 1000 by connecting it to the distal end of the endoscope. Furthermore, endoscopic cap 1010 may enable a user, e.g., a physician operating the endoscope to easily view the area of operation of the operating device passing through the external suction and work channel device 1000 via the scope and optics located at the distal end of the endoscope.

In some embodiments, external work and suction channel 1020 may comprise additional connectors 1014 to connect the external work and suction channel 1020 to an endoscope. Connectors 1014 may be located anywhere along external work and suction channel 1020. In some embodiments, connectors 1014 may be configured to enable the existing endoscope to pass therethrough, such to connect external work and suction channel 1020 to the existing endoscope. Connectors 1014 may be in the shape of a ring that is connected to external work/suction channel 1020, the ring being configured to enable passage of the endoscope therethrough. In other embodiments, other type of connectors 1004 and 1014 may be used.

In some embodiments, distal endoscopic cap 1010 may comprise a suction channel entry 1016 inside distal cap 1010, while configured to include additional space for insertion of the distal end of the existing endoscope into endoscopic cap 1010.

FIG. 11 is a schematic illustration of an external suction and work channel device attached to an endoscope, according to an embodiment of the disclosure. In some embodiments, accessory suction and work channel device 1000 may be releasably attachable to an existing endoscope 2000. In some embodiments, accessory suction and work channel device 1000 may be connected via connector 1004 and/or one or more connectors 1014 to an existing endoscope, e.g., endoscope 2000, which may comprise its own work channel 2010. In some embodiments, cap 1010 may also be configured to connect the distal end of accessory suction and work channel device 1000 to the distal end of the endoscope 2000.

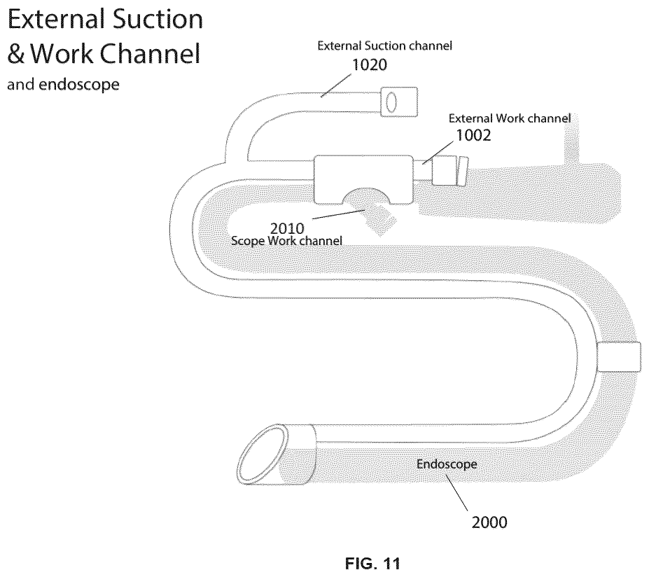

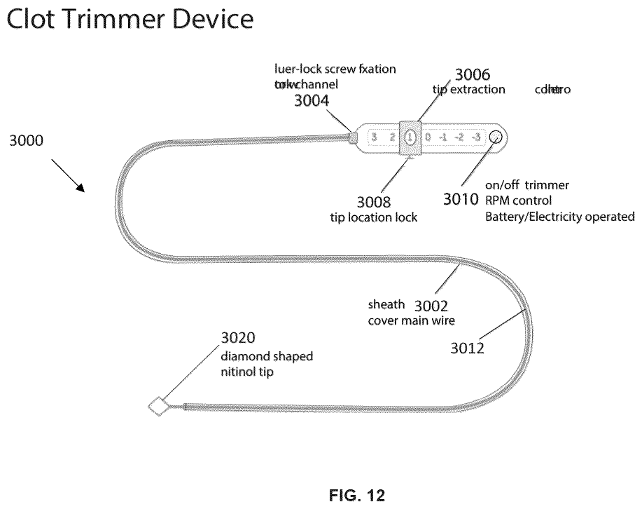

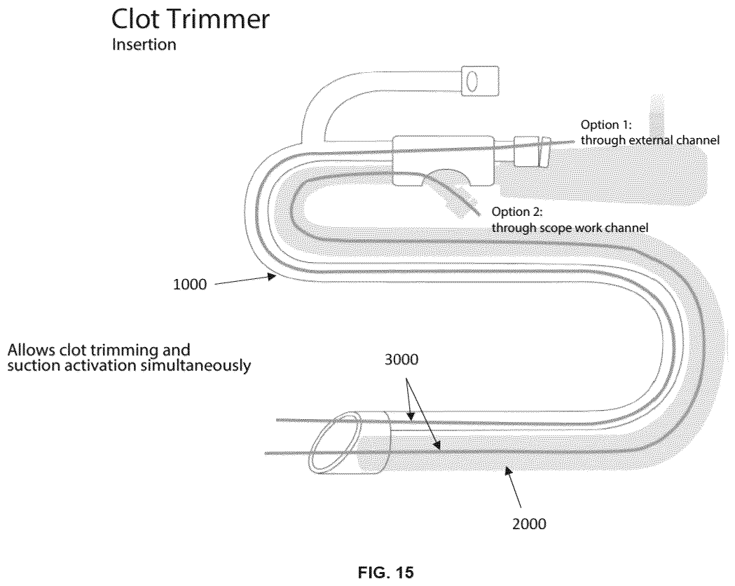

FIG. 12 is a schematic illustration of a blood clot trimmer device, according to another embodiment of the disclosure. In some embodiments, a blood clot trimmer device 3000 is provided. Optionally, blood clot trimmer device 3000 is configured to pass through the external suction and work channel device 1000, or through the working channel of an existing endoscope 2000, as illustrated in FIG. 15.

Adding accessory work/suction channel device 1000 to an existing endoscope provides an additional work channel, which may be used for simultaneous operation of two instruments in parallel, one passing through the existing endoscope's working channel, and the other passing through the accessory work/suction channel device 1000.

According to some embodiments, blood clot trimmer device 3000 may comprise a sheath 3002, which may cover guide wire 3012. Connected to guide wire 3012 at its distal end is clot trimmer tip 3020. In some embodiments, clot trimmer tip 3020 may be an integral part of guide wire 3012. In some embodiments, clot trimer tip 3020 may exit at the distal end of sheath 3002. In some embodiments, sheath 3002 may be made of polyethylene, though other materials may be used.

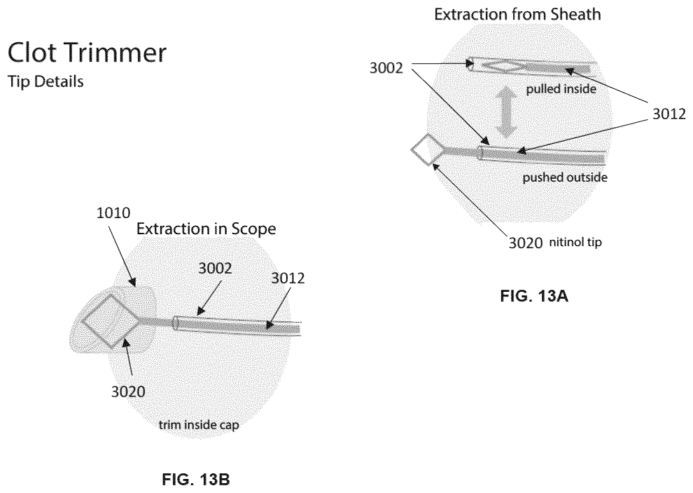

Optionally, clot trimmer tip 3020 may comprise a diamond shaped nitinol made tip which may act as a whisk configured to break apart blood clots while performing tip rotation. As illustrated in FIG. 13A, the guide wire 3012 may comprise a rigid wire, which may be made, for example, of metal, e.g., a stainless-steel braided wire, but could also be made of nitinol or other materials. Clot trimmer tip 3020 may comprise a flexible wire, which may be made, for example, of nitinol, though other materials may be used. Nitinol may be a preferrable material for tip 3020, for being super elastic and having shape memory properties, such that when retracted and passed through sheath 3002, tip 3020 is of a relatively flat configuration, whereas when extracted out of sheath 3002, tip 3020 regains its diamond shape, which proved to be highly efficient in trimming blood clots. The sheering force of the diamond shaped tip 3020 is able to break up blood clots efficiently, when coupled with the suction. In other embodiments, tip 3020 may implement other shapes such as an upside-down triangle (where the vertex of the triangle is located at the connection point between tip 3020 and the distal end of guide wire 3012), one or more circular shapes, etc. In some embodiments, tip 3020 may rotate to thereby break or cut blood clots during its rotation.

As illustrated in FIG. 12, blood clot trimmer device 3000 may further comprise a tip extraction controller 3006, which may control the extraction and/or the retraction of clot trimer tip 3020 outside and/or inside of the distal end of sheath 3002. Optionally, tip extraction controller 3006 may comprise a dial or a bar of numbers, which may indicate different possible distances of clot trimer tip 3020 either outside of the distal cap 1010 of accessory suction and work channel 1020 or inside the distal cap 1010 of accessory suction and work channel 1020. Each of the numbers along tip extraction controller 3006 may represent a movement of several mm, e.g., between 5 mm to 10 mm, though other distances may be implemented.

In some embodiments, tip extraction controller 3006 may comprise a tip location lock 3008, which a user, e.g., a physician, may move along the numbers representing distances on tip extraction controller 3006, to lock a specific distance to which the clot trimmer tip 3020 may be pushed (extracted) or pulled (retracted) out and in of accessory suction and work channel 1020. Tip location lock 3008 may be used to maintain the amount of retraction or extraction of clot trimmer tip 3020 with respect to either the distal cap 1010 or sheath 3002 or both, to be unchangeable, until tip location lock 3008 is moved again.

Location of tip 3020 with respect to distal end of sheath 3002, and typically within the perimeter of endoscopic cap 1010, should be unchanged during trimming operation, i.e., during rotation of tip 3020, for safety reasons, to avoid damage to tissue of the patient's lumen that is not a blood clot already sucked into endoscopic cap 1010 via external suction channel 1020. Such damage may occur if tip 3020 is extracted beyond the distal edges of endoscopic cap 1010.

According to some embodiments, tip extraction controller 3006 may comprise several positive numbers, a zero and several negative numbers. The positive numbers may indicate the degree of extraction of clot trimer tip 3020 outside of accessory suction and work channel 1020, the zero may indicate that clot trimer tip 3020 is located at the edge of distal cap 1010 of accessory suction and work channel 1020, and the negative numbers may indicate the degree of retraction of clot trimer tip 3020 inside of accessory suction and work channel 1020. Other markings, such as numbers from zero and above, may be used to indicate retraction and extraction of clot trimer tip 3020 in and out of accessory suction and work channel 1020.

In some embodiments, sheath 3002 may comprise fixation means 3004, e.g., a screw, or luer lock, on the proximal end of sheath 3002 or along the proximal end of tip extraction controller 3006. Fixation means 3004, which may be made of plastic, such as Polycarbonate, may be used to fixate sheath 3002 to the accessory suction and work channel 1020, whereby the accessory suction and work channel 1020 is connected to the endoscope 2000. Blood clot trimmer device 3000 may then be inserted into a site internal to a patient body, e.g., the gastrointestinal tract, via accessory suction and work channel 1020. Fixation means 3004 may be used to create a tight connection between sheath 3002 and accessory suction and work channel 1020 such that operation of suction through accessory suction and work channel 1020 is sufficient.

In some embodiments, where a motor is implemented as part of blood clot trimmer device 3000, the motor unit may also be fixated to accessory work/suction channel 1020 via fixation means 3004.

In some embodiments, blood clot trimmer device 3000 may further comprise an on/off operation mechanism 3010, e.g., a press button, which may initiate or cease operation of blood clot trimmer device 3000 and may further control revolutions per minute or speed of rotation of the clot trimmer tip 3020, e.g., by controlling operation of a motor connected to tip 3020. Optionally, blood clot trimmer device 3000 may comprise a power supply (not shown), e.g., a battery, a rechargeable battery or an electricity connection, which may be operated by pressing button 3010. When the power supply is operated, it may cause operation of a motor which rotates trimmer tip 3020 and thus enable trimming and breaking of blood clots. Typically, a battery-operated device is less cumbersome and easier to manipulate while manipulating an existing endoscope.

FIGS. 13A-13B are schematic illustrations of a blood clot trimmer device tip 3020 when the clot trimmer tip 3020 is inside the sheath 3002, when the tip 3020 is outside the sheath 3002, and when the tip 3020 is outside the sheath 3002 but within distal endoscopic cap 1010, respectively, according to an embodiment of the disclosure. FIG. 13A illustrates trimmer tip 3020 that is connected to a guide wire 3012. In some embodiments, sheath 3002 is configured to cover guide wire 3012. That is, as illustrated in FIG. 13A, guide wire 3012 is pulled back into sheath 3002, e.g., by locking tip location lock 3008 on tip extraction controller 3006 on a number representing a retraction position, e.g., a negative number. On the contrary, FIG. 13A illustrates guide wire 3012 pushed outside of sheath 3002, which causes tip 3020 to be extracted outside of sheath 3002, for example, to engage a trimming process.

FIG. 13B illustrates tip 3020 after it is extracted from sheath 3002 but still located within the space of distal endoscopic cap 1010. Locating trimmer tip 3020 at the distal edge of endoscopic cap 1010 and not outside of perimeter of cap 1010, provides a safer operation of trimmer tip 3020, thereby avoiding damage to surrounding tissue during rotation of trimmer tip 3020. That is, only blood clots that are sucked into endoscopic cap 1010 via accessory suction and work channel 1020 would be trimmed by trimmer tip 3020. However, in some embodiments, a user may extract tip 3020 further out of endoscopic cap 1010, e.g., in order to trim blood clots that are difficult to reach via the endoscopic cap 1010 alone.

According to some embodiments, the size of endoscopic cap 1010 is longer than the length of trimming tip 3020 to avoid trimming tip 3020 from contacting patients tissue.

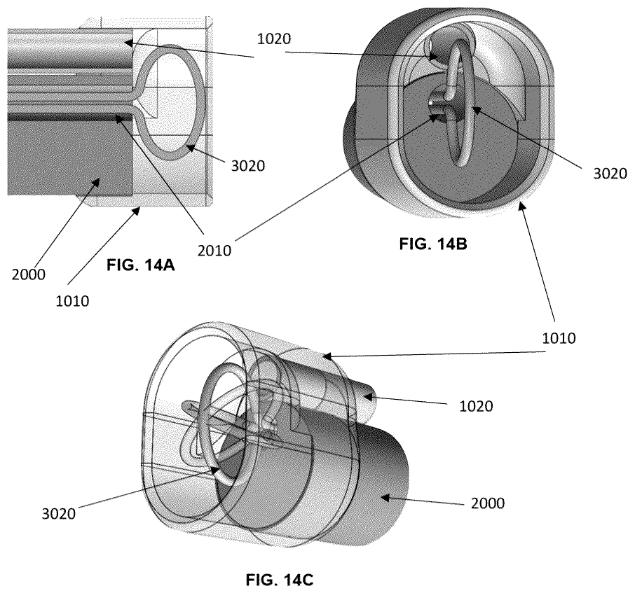

FIGS. 14A-14B are schematic illustrations of a cross-section view and a front-perspective view, respectively, of a tip of a blood clot trimmer device with its tip extracted outside of the sheath and within the endoscopic cap, according to an embodiment of the disclosure. In some embodiments, blood clot trimmer device 3000 may be passed along an existing endoscope's working channel 2010. In some embodiments, blood clot trimmer device tip 3020 may be pushed or extracted outside of sheath 3002 while still being located within the perimeter of endoscopic cap 1010.

As illustrated in FIGS. 14A-14B, guide wire 3012 may be pushed outside of endoscope working channel 2010, such that blood clot trimmer device tip 3020 is extracted outside of endoscope working channel 2010, while maintained within endoscopic cap 1010. In such case, external work/suction channel 1020 may be used solely for suction.

FIG. 14C is a schematic illustration of a perspective view of the blood clot trimmer tip 3020 during rotation process.

During operation of blood clot trimming, suction is operated via external work/suction channel 1020 to suck a blood clot that is present in the internal site of a patient's body, e.g., the upper gastrointestinal tract, into endoscopic cap 1010. Then, blood clot trimmer device tip 3020 may rotate to trim and break the blood clot that is sucked into endoscopic cap 1010, without causing any harm to surrounding tissue, due to the flexibility characteristics of tip 3020, as well as its location within endoscopic cap 1010.

In some embodiments, the speed of rotation of trimmer tip 3020 may be controlled by the user.

In some embodiments, when blood clot trimmer device tip 3020 is located inside the distal end of endoscopic cap 1010, after tip 3020 is extracted from sheath 3002, tip 3020 may be operated such to rotate and trim a blood clot, when blood clot trimmer device tip 3020 is pressed against the blood clot's surface or edges. In some cases, such trimming is a more focused trimming, which may cause less harm to tissue surrounding the blood clot.

In other embodiments, suction is first applied via accessory suction and work channel 1020 to suck a blood clot into endoscopic cap 1010, and only then is tip 3020 rotated to simultaneously trim the blood clot by tip 3020 while using the suction to remove the trimmed pieces outside of the internal site in the patient's body all at once.

In standard endoscopes, when something occupies the endoscope's working channel, suction through the endoscope's working channel may not be used or is greatly reduced. However, since accessory work/suction channel 1020 operates as a suction channel that is independent from the endoscope's working channel, suction would be operated regardless of what may occupy or obstruct the endoscope's working channel, e.g., the blood clot trimmer device passing through the endoscope's working channel. That is, accessory work/suction channel 1020 provides an independent suction/vacuum system that is in addition to the working channel of the endoscope, and thus its operation is highly efficient compared to using suction through the existing endoscope's working channel.

In addition, simultaneous suction and trimming is possible in by the blood clot trimmer device of the present disclosure, even when the blood clot trimmer device passes through the external work/suction channel 1020 due to the blood clot trimmer device being inserted into the external suction\working channel which is wide in diameter and is connected to direct suction, so the blood clot trimmer device does not obstruct suction efficacy.

In some embodiments, the rotating diamond shaped clot trimmer tip 3020 may be pulled into sheath 3002 when not in use, to clear the view of endoscope 2000, and for safe transportation of blood clot trimmer device 3000 along the working channel 2010 of endoscope 2000. Instead, tip 3020 may be advanced out of the distal end of sheath 3002 to open into its diamond shape and be functionally used inside endoscopic cap 1010, for trimming blood clots.

FIG. 15 is a schematic illustration of two options through which a blood clot trimmer device 3000 can pass, one is through an external suction and work channel device 1000 and a second is through an existing work channel of an endoscope 2000. In some embodiments, blood clot trimmer device 3000 may be passed through an external suction and work channel device 1000. Whereas, in other embodiments, blood clot trimmer device 3000 may be passed through an existing work channel or an existing endoscope, e.g., through work channel 2010 of endoscope 2000.

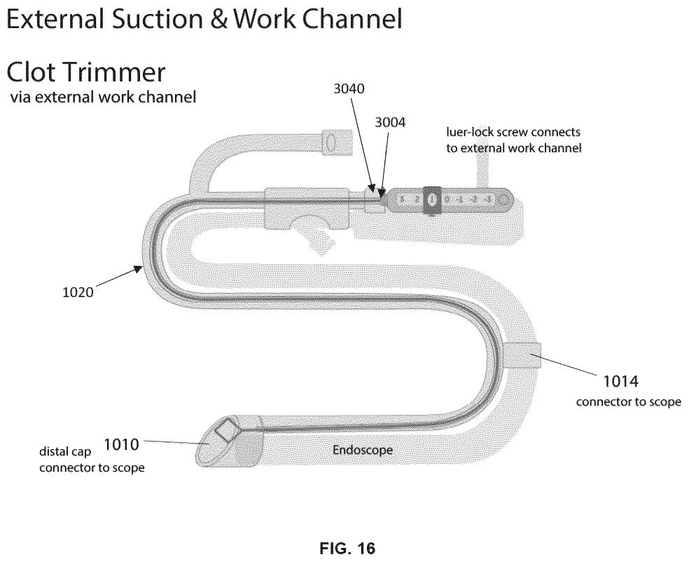

FIG. 16 is a schematic illustration of the blood clot trimmer device passing through the external work channel device, according to an embodiment of the disclosure. According to some embodiments, blood clot trimmer device 3000 may pass through an external work channel device 1000, in order to provide blood clot trimming capabilities without occupying an existing work channel of an existing endoscope 2000. As explained with respect to FIG. 10, external work channel device 1000 may be connected to an endoscope 2000 via a connector 1004 and possibly via endoscopic cap 1010. And as explained with respect to FIG. 12, and as illustrated in FIG. 16, blood clot trimmer device 3000 may pass through an external work channel device 1000, while being connected to the accessory work channel 1020 via fixation means 3004, which may comprise luer lock connection/fixation means 3004. That is, tip extraction controller 3006 may comprise a luer lock 3004 surrounding the passage which the sheath 3002 and guide wire 3012 pass therethrough, and this luer lock 3004 may be screwed onto a corresponding luer lock lip 3040 at the proximal end of the external work channel cap 1002, to properly fixate blood clot trimmer device 3000 to the external suction and work channel 1000.

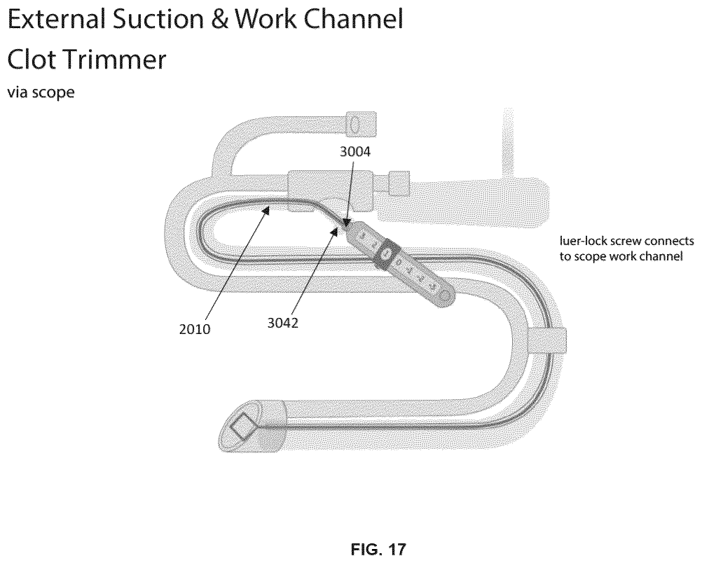

FIG. 17 is a schematic illustration of the blood clot trimmer device 3000 passing through an existing work channel of an endoscope, according to an embodiment of the disclosure. According to some embodiments, blood clot trimmer device 3000 may be fixated to the endoscope's work channel, e.g., via fixation means 3004, which may comprise luer lock connection means. That is, tip extraction controller 3006 may comprise a luer lock 3004 surrounding the passage which the sheath 3002 and guide wire 3012 pass therethrough, and this luer lock 3004 may be screwed onto a corresponding luer lock lip 3042 at the proximal end of the existing work channel 2010, to properly fixate blood clot trimmer device 3000 to the existing work channel 2010.

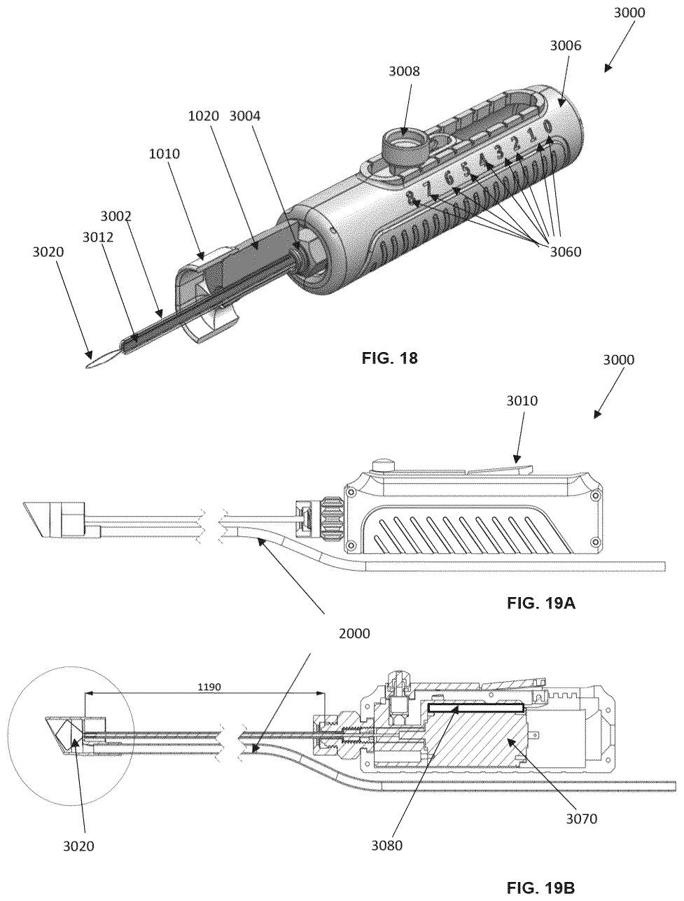

FIG. 18 is a schematic perspective view of the blood clot trimmer device 3000, from handle to tip, according to an embodiment of the disclosure. In some embodiments, blood clot trimmer device 3000 may comprise a handle which may be a tip extraction controller 3006, configured to control retraction/extraction state of trimmer tip 3020. A explained hereinabove, trimmer tip 3020 may be an integral part of or connected to guide wire 3012, which may be covered by and may pass through sheath 3002. In order for a user to determine the length of guide wire 3012 that is extracted outside of sheath 3002, or to determine the extent of retraction of guide wire 3012 inside sheath 3002, the user may move a tip location lock 3008 along scale marks 3060 illustrated on tip extraction controller 3006. Each of the scale marks 3060 of the tip extraction controller 3006, may indicate a length size, e.g., 5 m to 10 mm. Thus, once a user moves the tip location lock 3008 along the scale marks 3060, the user determines whether the tip 3020 is retracted into sheath 3002 or extracted out of sheath 3002. Scale marks 3060 may include numbers from zero and up, whereas in other embodiments, the scale marks 3060 may include negative numbers, zero and positive numbers, each indicating location of tip 3020 with respect to the distal end of sheath 3002.

In some embodiments, tip location lock 3008 may be turned or otherwise fixated along tip extraction controller 3006 at a certain scale mark 3060, such to lock the determined location of tip 3020 with respect to the distal end of sheath 3002.

FIGS. 19A-19B are schematic illustrations of the blood clot trimmer device 3000, in side-view and cross-section view, respectively, according to an embodiment of the disclosure. In some embodiments, blood clot trimmer device 3000 may comprise a power mechanism 3010, which may be an ON/OFF operation button. The ON/OFF operation mechanism 3010, may be a press button, which may initiate or cease operation of blood clot trimmer device 3000, and may further control revolutions per minute or speed of rotation of the clot trimmer tip 3020. Optionally, blood clot trimmer device 3000 may comprise a power supply 3080 e.g., a battery, a rechargeable battery, an AC adaptor, or an electricity connection, which may be operated by pressing button 3010.

In some embodiments, blood clot trimmer device 3000 may comprise a motor 3070, which may be powered by the power supply 3080 of blood clot trimmer device 3000. Motor 3070 may be used to transform electrical power to mechanical movement, i.e., rotation of trimmer tip 3020. When power supply 3080 is operated, it causes the motor 3070 to rotate trimmer tip 3020 and thus enable trimming and breaking of blood clots. In some embodiments, motor 3070 may spin at a maximum of 8000 rpm, though other velocities may be implemented.

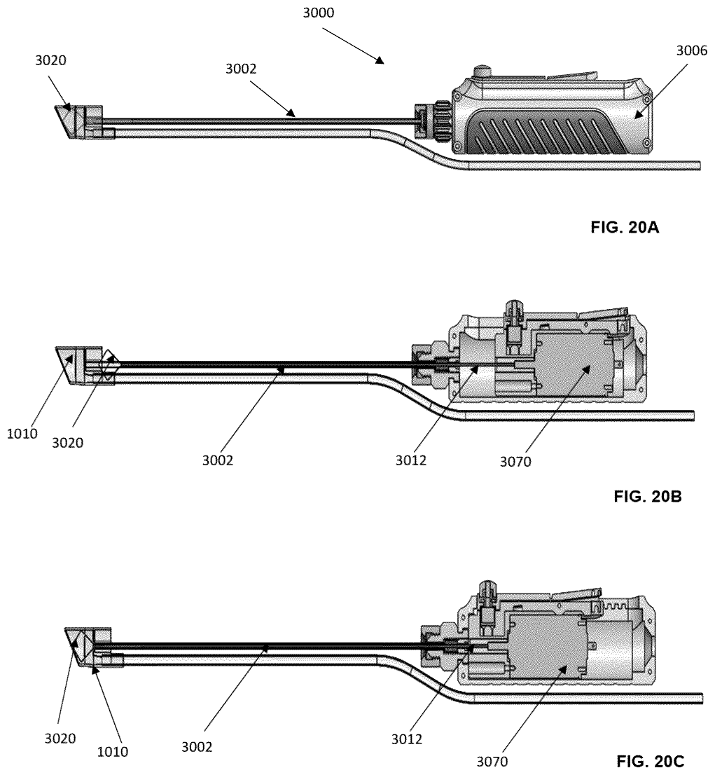

FIGS. 20A-20C are schematic illustrations of a motorized blood clot trimmer device in side-view, cross-section view when the trimmer tip is retracted from the endoscopic cap, and a cross-section view when the trimmer tip is extracted and positioned within the endoscopic cap, respectively, according to embodiments of the disclosure. In some embodiments, the blood clot trimmer device 3000 may be a motorized blood clot trimmer device, which comprises motor unit 3070. Motor unit 3070 is connected to guide wire 3012, such that movement of motor 3070 is equivalent to movement of guide wire 3012 within sheath 3002. When motor 3070 is moved within the case of tip extraction controller 3006, towards the proximal end of tip extraction controller 3006, guide wire 3012 is retracted into sheath 3002 and pulled towards the proximal end of blood clot trimmer device 3000, as illustrated in FIG. 20B. Whereas, when motor 3070 is pushed within the casing of tip extraction controller 3006, towards the distal end of tip extraction controller 3006, guide wire 3012 may be extracted from the distal end of sheath 3002, ready for rotation within endoscopic cap 1010.

According to some embodiments, motor 3070 may be reusable, while the sheath 3002 and guide wire 3012 may be detachable from motor 3070 and may be disposable, i.e., disregarded after use.

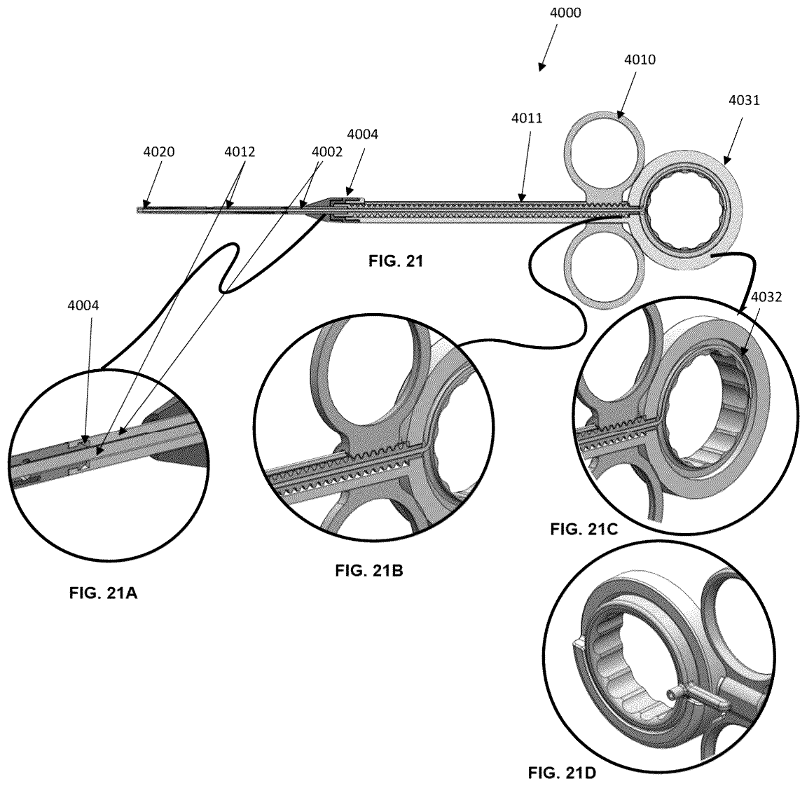

FIGS. 21-21D are schematic illustrations of a manual blood clot trimmer device and enlarged views of the manual blood clot trimmer's circular connection, linear to radial movement, and the guide wire retraction mechanism, respectively, according to an embodiment of the disclosure. In some embodiments, the blood clot trimmer device may be a manual device 4000 instead of a motorized blood clot trimer device 3000. Accordingly, blood clot trimmer device 4000 may not comprise a motor but rather a manual mechanism that enables rotation of trimmer tip 4020 for cutting and trimming blood clots and enabling determination of extraction/retraction degree of tip 4020 with respect to the distal end of sheath 4002. Trimmer tip 4020 may be similar to trimmer tip 3020, and may also comprise a diamond shaped tip, which may be made of an elastic material, which may also comprise shape memory properties, e.g., nitinol. Trimmer tip 4020 may be an integral part or may be connected to guide wire 4012, which may pass through sheath 4002.

In some embodiments, as illustrated in FIG. 21A, blood clot trimmer device 4000 may comprise a circular connector, or fixation means 4004, which may be similar to fixation means 3004. That is, fixation means 4004 may comprise a luer lock to connect the manual blood clot trimmer device 4000 to either the external work/suction channel 1020 or the working channel 2010 of an existing endoscope 2000, both of which may comprise corresponding luer lock lip means.

In some embodiments, as illustrated in FIG. 21B, blood clot trimmer device 4000 may comprise a linear to radial movement translator mechanism. The linear to radial translator mechanism may comprise a threaded rod 4011 along which threaded handle 4010 and handle end 4031 may rotate, thereby causing guide wire 4012 to enter or exit an inner circular channel 4032 within handle end 4031, which thereby causes trimmer tip 4020 to be extracted or retracted with respect to sheath 4002. Threaded handle 4010 may comprise two circular openings configured for placement of fingers of a user, typically each circular opening is configured for operation by one finger. When turning threaded handle 4010 in one direction by fingers of the user, e.g., clockwise, guide wire 4012 is pulled into inner channel 4032, thereby retracting tip 4020 into sheath 4002. However, when turning threaded handle 4010 in the opposite direction, e.g., counterclockwise, guide wire 4012 is pushed out of inner channel 4032, thereby extracting tip 4020 out of sheath 4002. FIGS. 21C-21D illustrate the guide wire 4012 retraction mechanism into inner channel 4032, which occurs automatically during rotation of threaded handle 4010.

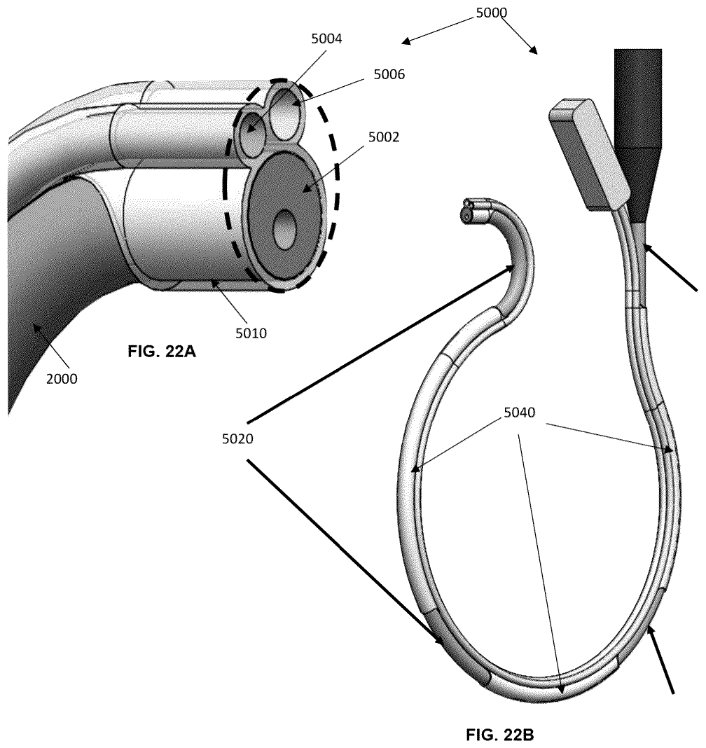

FIGS. 22A-22B are schematic illustrations of a multi-lumen accessory suction and work channel, according to embodiments of the disclosure. In some embodiments, the accessory work and suction channel 5000 may comprise of two accessory channels or two lumens, connected to an endoscope 2000 via a distal endoscopic cap 5010. The endoscopic cap 5010 may comprise an opening for insertion of and attachment to the distal end of an existing endoscope 2000. Distal endoscopic cap 5010 may further be attached to two work channels, first work channel 5004 may be configured to allow passage of a working instrument, such as a blood clot trimmer device, e.g., blood clot trimmer device 3000 or 4000. A second work channel 5006 may be configured to operate as a suction channel.

In some embodiments, the diameter of the endoscopic cap 5010 that is placed around the distal end of the endoscope 2000 may be about 9 mm, which fits a standard endoscope's diameter. In some embodiments, accessory work channel 5004 may have a diameter of about 3 mm to 4 mm, e.g., 3.15 mm, suitable for passage of GI related instruments therethrough. In some embodiments, accessory suction channel 5006 may have a diameter of around 3 mm to 4 mm, 3. g., 3.7 mm, suitable for passage of relatively large particles. Thus, the total diameter of the multi-lumen accessory work and suction channel 5000 may be around 15 mm.

FIG. 22B schematically illustrates a full-sized multi-lumen accessory work and suction channel device 5000. In some embodiments, multi-lumen accessory work and suction channel 5000 may comprise connection areas 5040 between the accessory work and suction channel 5000 and the endoscope 2000 to which the multi-lumen accessory work and suction channel 5000 is releasably attached. Between those connection areas 5040 there may be relief areas 5020 to enable sufficient flexibility of the endoscope 2000 for ease of maneuvering endoscope 2000 during its operation. In some embodiments, the relief areas 5020 may be of a length of approximately 80 mm, with a length of approximately 160 mm separating between each two sequential relief areas. In other embodiments, other lengths for the relief areas 5020 and the separating areas (which may also operate as connection areas 5040 between the multi-channel accessory work and suction channel 5000 and endoscope 2000) may be implemented.

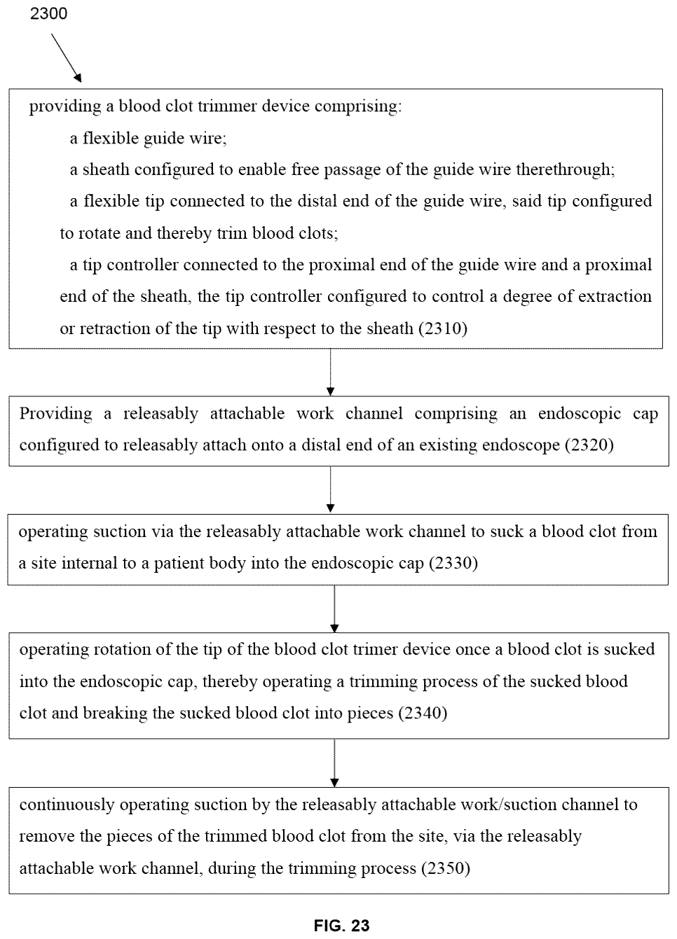

FIG. 23 is a flow chart schematically illustrating a method for operating the blood clot trimmer device, according to an embodiment of the disclosure. In some embodiments, method 2300 may comprise operation 2310, which may comprise providing a blood clot trimmer device comprising: (i) a flexible guide wire including a proximal end and distal end; (ii) a sheath configured to enable free passage of the guide wire therethrough, said sheath including a proximal end and a distal end; (iii) a flexible tip connected to the distal end of the guide wire, said tip configured to rotate and thereby trim blood clots; and (iv) a tip controller connected to the proximal end of the guide wire and a proximal end of the sheath, the tip controller configured to control a degree of extraction or retraction of the tip with respect to the sheath.

In some embodiments, method 2300 may further comprise operation 2320, which may comprise providing a releasably attachable work channel comprising an endoscopic cap configured to releasably attach onto a distal end of an existing endoscope. In some embodiments, the blood clot trimmer device may pass through the releasably attachable work channel, while in other embodiments, the blood clot trimmer device may pass through a working channel of an existing endoscope.

In some embodiments, method 2300 may comprise operation 2330, which may comprise operating suction via the releasably attachable work channel to suck a blood clot from a site internal to a patient body into the endoscopic cap.

In some embodiments, method 2300 may further comprise operation 2340, which may comprise operating rotation of the tip of the blood clot trimer device once a blood clot is sucked into the endoscopic cap, thereby operating a trimming process of the sucked blood clot and breaking the sucked blood clot into pieces. And in some embodiments, method 2300 may further comprise operation 2350, which may comprise continuously operating suction by the releasably attachable work/suction channel to remove the pieces of the trimmed blood clot from the site, via the releasably attachable work channel, during the trimming process.

In some embodiments, method 2300 may comprise moving a tip location lock along scale marks on the tip controller, thereby determining the degree of extraction or retraction of the tip with respect to the distal end of the sheath.

In some embodiments, method 2300 may further comprise locking the determined position of the tip with respect to the distal end of the sheath to prevent further movement of the tip with respect to the sheath.

In some embodiments, the blood clot trimmer device comprises a motor, thus method 2300 may comprise causing movement of the guide wire and the tip connected to the guide wire with respect to the sheath by moving the motor, thereby determining degree or extraction or retraction of the tip with respect to the distal end of the sheath and operating the motor for rotating the tip.

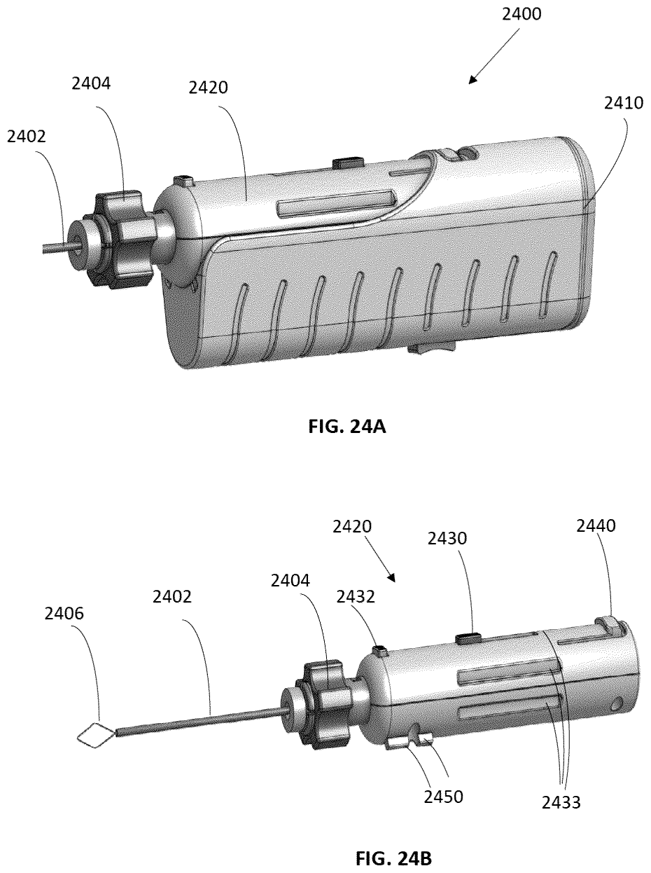

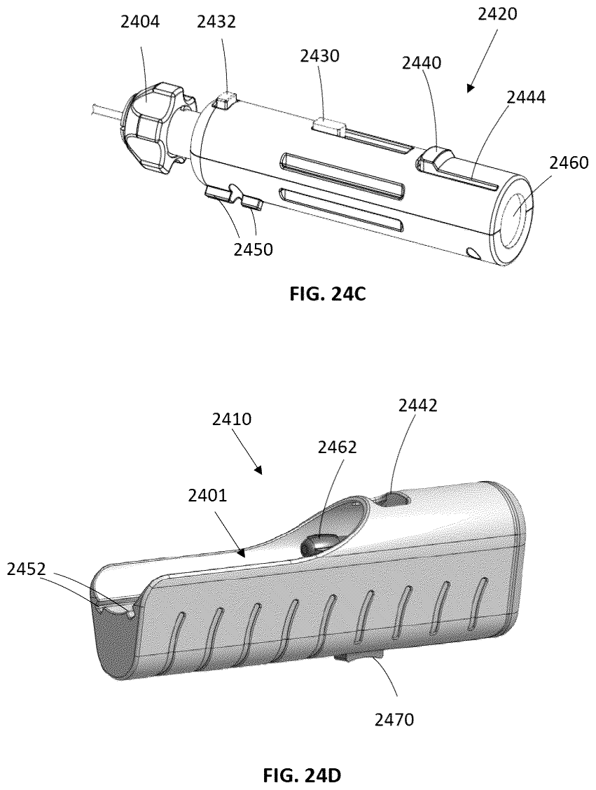

Reference is now made to FIGS. 24A-24D, which are schematic illustrations of a side-perspective view of the two-part handle of the blood clot trimmer device, a side-perspective view of the disposable unit of the handle, a rear-perspective view of the disposable unit of the handle, and a side-perspective view of the reusable unit of the handle, according to embodiments of the present disclosure. According to some embodiments, the motorized blood clot trimmer device of the present disclosure, e.g., blood clot trimmer device 300 or 3000, may comprise a two-part handle 2400 to replace the handle described hereinabove. Two-part handle 2400 may comprise a reusable unit 2410, and a disposable unit 2420. Reusable unit 2410 may comprise the motor and power source, which may be completely sealed and thus repeatedly used for multiple procedures. Whereas the disposable unit 2420, which includes at least the sheath 2402, the guide wire passing therethrough (not shown) and the fixation means 2404, is disposable, such to ensure that all the elements inserted into the gastrointestinal tract are new and sterile per each procedure in which the blood clot trimmer device is used. This two-part handle is both cost effective and minimizes waste, since part of it is reusable while the other disposable.

In some embodiments, device 2420 may comprise fixation means 2404, e.g., a screw, or luer lock, on the proximal end of disposable unit 2420. Fixation means 2404, which may be made of plastic, such as Polycarbonate, may be used to fixate device 2400, via disposable unit 2420, to the accessory suction and work channel 1020, whereby the accessory suction and work channel 1020 is connected to the endoscope or colonoscope 2000. Blood clot trimmer device 300 or 3000 may then be inserted into a site internal to a patient body, e.g., the gastrointestinal tract, whether the upper GI or lower part of it, e.g., the colon, via accessory suction and work channel 1020. Fixation means 2404 may be used to create a tight connection between disposable unit 2420 and accessory suction and work channel 1020 such that operation of suction through accessory suction and work channel 1020 is sufficient.

In some embodiments, disposable unit 2420 may comprise a slidable button 2430, which is configured to control the degree of extraction and/or retraction of clot trimmer tip 2406 from and into sheath 2402. Movement of slidable button 2430 is enabled along indentations or rails 2433. When slidable button 2430 is moved towards the proximal end of handle 2400, the longer clot trimmer tip 2406 is, i.e., clot trimmer tip 2406 is extracted from sheath 2402. However, when slidable button 2430 is moved towards the distal end of handle 2400, clot trimmer tip 2406 is pulled into sheath 2402.

The lengths of the sheath 2402 and of the guide wire (not shown) connected to the clot trimmer tip 2406 (similar to guide wire 3012 in FIG. 18) may be predetermined such to correspond to the size of the endoscope, gastroscope or colonoscope that the blood clot trimmer device is inserted therethrough. For example, for a blood clot trimmer device that should be inserted into a colonoscope, the length of the sheath, e.g., from the distal clot trimmer tip 2406 to the working channel, should be approximately 1800 mm-1870 mm. However, other lengths may be implemented, to fit any type of gastrointestinal medical device.

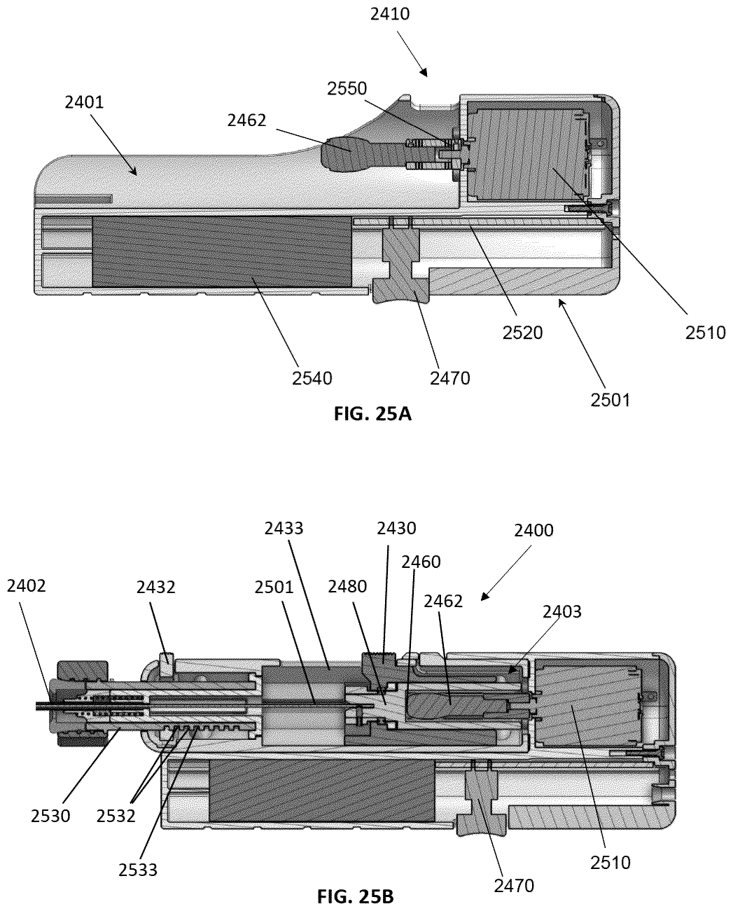

In some embodiments, disposable unit 2420 may further comprise an additional button 2432, which may be used to provide additional small ranges of lengths, per extraction and retraction of the sheath 2402, the guide wire and clot trimmer tip 2406 from and into disposable unit 2420, to overcome any manufacturing variances, as will be explained in detail with respect to FIG. 25B.

In some embodiments, disposable unit 2420 may comprise guiding protrusions 2450, which may be configured to be inserted into corresponding indents 2452 configured to accept the guiding protrusions 2450 therein. Such guiding protrusions 2450 and their corresponding indents 2452, may ensure that the disposable unit 2420 is positioned at a certain location with respect to reusable unit 2410, and avoid rotation of disposable unit 2420 within the housing 2401 of reusable unit 2410 during operation of the motor in reusable unit 2410, which rotates the clot trimmer tip 2406. Thus, rotation of the motor would only cause rotation of clot trimmer tip 2406 and would not cause rotation of the entire disposable unit 2420, which would interrupt with proper operation of the clot trimmer tip 2406. The only element that should be rotating during a blood clot trimming procedure is clot trimmer tip 2406, and not the entire disposable unit 2420, which guiding protrusions 2450 and their respective indents 2452 ensure.

According to some embodiments, disposable unit 2420 may further comprise a snap 2440. Snap 240 is pushed and snaped into a corresponding hole 2442. Snap 2440 is configured to ensure that disposable unit 2420 is fitted within and hugged by housing 2401 of reusable unit 2410.

When a user wishes to connect disposable unit 2420 to reusable unit 2410, the user pushes or slides disposable unit 2420 into housing 2401, such that snap 2440 is pushed against the walls of housing 2401 that create hole 2442, until snap 2440 reaches hole 2442. In case disposable unit 2420 is already connected to a gastrointestinal device, e.g., an endoscope, colonoscope, etc., via fixation means 2404, the reusable unit 2410 may be slid into disposable unit 2420, such to connect between reusable unit 2410 and disposable unit 2420. Typically, snap 2440 may include two slots 2444, located longitudinally on both sides of snap 2440 to provide some elasticity to snap 2440 and allow snap 2440 to rise back to its initial position after it is depressed or pushed downwards. Accordingly, when snap 2440 is pushed beneath the walls of housing 2401, which surround hole 2442, snap 2440 is pushed downwards, however, when snap 2440 reaches hole 2442, such that snap 2440 can no longer be pushed further into housing 2401, snap 2440 automatically rises upwards, due to its elasticity, such to fill hole 2442 and protrude a bit outside of hole 2442. This snapping prevents spontaneous disconnection of disposable unit 2420 from reusable unit 2420. And when a user wishes to manually disconnect disposable unit 2420 from reusable unit 2410, e.g., before discarding disposable unit 2420, the user presses downwards on snap 2440 such to push snap 2440 into hole 2442, and the user further pulls disposable unit 2420 away from housing 2401, to thereby separate disposable unit 2420 from reusable unit 2410.

In some embodiments, disposable unit 2420 may further comprise a groove 2460 located at the proximal end of disposable unit 2420. Groove 2460 may be configured to accept therein a corresponding shaft 2462, which is part of reusable unit 2410. The corresponding groove 2460 and shaft 2462 may be configured to ensure proper connection between clot trimmer tip 2406 and the motor 2510 (see FIGS. 25A-25B) housed within reusable unit 2410. The element within disposable unit 2420 which comprises groove 2460 is configured to transfer motor rotation movement to the guide wire, as will be explained in detail with respect to FIGS. 25A-25B.

In some embodiments, shaft 2462 may be a serrated shaft, which may comprise protrusions along its distal end, whereby those protrusions may be configured to be inserted into corresponding indentations optionally present along groove 2460. In such embodiments, the protrusions of the serrated shaft 2462 may dictate a specific position of the disposable unit 2420 with respect to reusable unit 2410 and provide better engagement between serrated shaft 2462 and groove 2460, which is equivalent to better engagement between clot trimmer tip 2406 and the motor. Accordingly, rotation of clot trimmer tip 2406 is enabled by the motor, via rotation of shaft 2462 within groove 2460.

Reference is now made to FIGS. 25A-25B, which are schematic illustrations of a cross-section of the reusable unit, and of the two-part handle of the blood clot trimmer device, respectively, according to embodiments of the present disclosure. In some embodiments, reusable unit 2410 may comprise a main housing 2401, which is configured to house disposable unit 2420 therein. In some embodiments, reusable unit 2410 may comprise a rear housing or rear shell 2501, which is configured to house the various components of reusable unit 2410. According to some embodiments, rear housing 2501 may include a motor 2510, which may rotate to thereby cause rotation of clot trimmer tip 2406. In some embodiments, motor 2510 is operated by power source 2540, to rotate, which in turn causes rotation of shaft 2462, since shaft 2462 is connected to motor 2510. When disposable unit 2420 is connected to reusable unit 2410, shaft 2462 is inserted into and connected to the walls of groove 2460. Groove 2460 is in fact a groove created within part 2480, which may rotate within housing 2403 of disposable unit 2420. Part 2480, which comprises groove 2460, is attached to guide wire 2501, which is in turn connected, and in some cases, is an integral part of clot trimmer tip 2406. Thus, rotation of shaft 2462 within groove 2460 causes rotation of part 2480, which then rotates clot trimmer tip 2406 via the direct connection between part 2480 and guide wire 2501.

According to FIG. 25, it can be seen that slidable button 2430 may slide along rail 2433 on both directions, such to move guide wire 2501, either towards the distal end of two-part handle 2400 or towards the proximal end of two-part handle 2400, thereby controlling extraction and retraction of clot trimmer tip 2406 out and in of sheath 2402.

As further illustrated by FIG. 25B, additional button 2432 may snap into different notches 2533, each located in between teeth 2532, which are all part of guide 2530. Sheath 2402 and guide wire 2501 pass through guide 2530. Button 2432 is configured to snap into either of the notches 2533 along guide 2530, such to change the retraction and extraction degree of sheath 2402 with respect to the distal end of the endoscope or colonoscope (or any other GI device). Button 2432 as illustrated in FIG. 25B is located at its most retracted position, by being snapped into the most distal notch 2533. In case a user wishes to change the extraction degree of sheath 2402 from disposable unit 2420, the user may press down on button 2432 to release it from the notch it is currently snapped into, and the user may extend guide 2530 in a forward direction, i.e., towards the distal end of disposable unit 2420, and when extension of guide 2530 is finished, button 2432 is snapped into a new notch, e.g., located at the most proximal end of guide 2530, which is equivalent to sheath 2402 being extracted from disposable unit 2420 to its maximum extraction distance. The teeth 2532, which notches 2533 are created therebetween, provide stability to the desired degree of extraction/retraction of sheath 2402, once the desired length of guide 2530 is selected. That is, teeth 2532 prevent button 2432 from changing its position, until button 2432 is pressed to enable such movement. Thus, guide 2530 acts as a neck that may extend or retract to thereby extend and retract sheath 2402 along with guide wire 2501, up to 30 mm.

A user may use button 2432 for additional extraction or retraction of sheath 2402 to overcome variances in manufacturing per different scopes. That is, even within the same type of GI imaging devices, there may be slight variation in the length of the working channel, e.g., due to repair. Thus, this additional ability to further adjust the length of sheath 2402 with respect to the working channel of the GI device, may be beneficial.

In some embodiments, reusable unit 2410 comprises a power source 2540, which may be one or more batteries. Power source 2540 may be a rechargeable battery. In such case, reusable unit 2410 may comprise a USB-C port (not shown) for recharging the battery therethrough.

In some embodiments, reusable unit 2410 may comprise PCB 2520, which may be connected to all of the electrical components of reusable unit 2410.

In some embodiments, reusable unit 2410 may comprise a switch 2470, which may be an ON/OFF switch for operating or ceasing operation of motor 2510. Switch 2470 may be a momentary operation type switch, which turns ON motor 2510 when switch 2470 is pushed, and turns motor 2510 OFF when switch 2470 is released. Switch 2470 may also be a standard ON/OFF switch, such that when the switch is pushed, the motor 2510 begins to rotate and the user need not continuously apply pressure on the switch, as opposed to momentary operation, and when the switch is pressed again, motor 2510 is turned OFF.

In some embodiments, reusable unit 2410 may further comprise an indication LED (not shown) configured to indicate status of motor 2510, i.e., whether motor 2510 is switched ON or OFF. Indication LED may comprise at least one illumination source of a single color to indicate ON or OFF state, while in other cases may include an additional LED of a different color, such that one LED of a first color would indicate the ON status of motor 2510, while the other LED of a second color would indicate the OFF status.

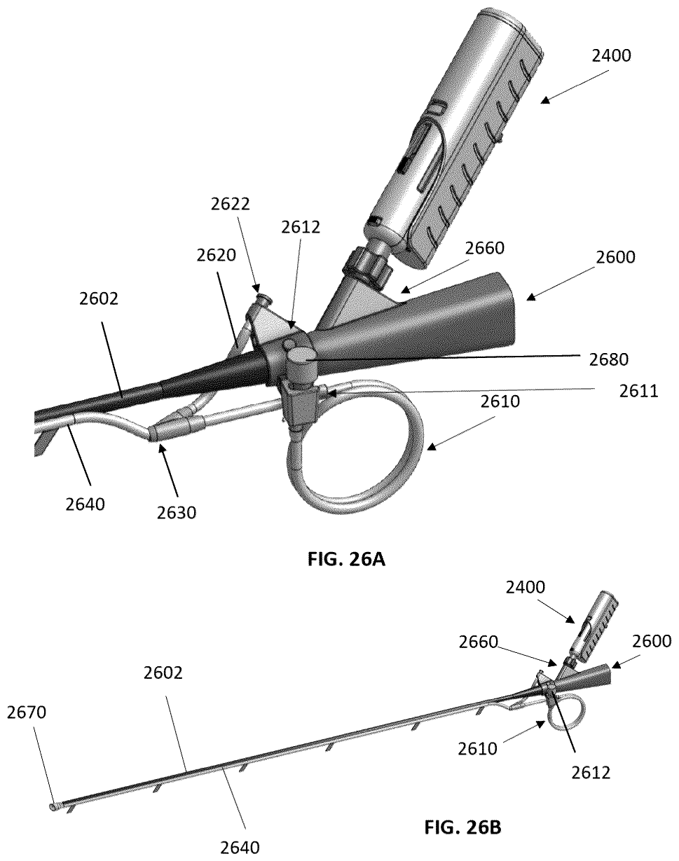

Reference is now made to FIGS. 26A-26B, which are schematic illustrations of an enlarged side-perspective view and a side-perspective view, respectively, of a blood clot trimmer, according to embodiments of the present disclosure. In some embodiments, the blood clot trimmer device may be inserted either through an existing working channel of an existing endoscope or colonoscope, or through an external working channel attached to the existing endoscope or colonoscope. In FIGS. 26A-26B, the working channel, which is part of the gastrointestinal imaging device 2600 is illustrated as working channel 2660. Two-part handle 2400 is inserted through working channel 2660.

In some embodiments, suction means may be connected to the blood clot trimmer device via an external connection 2612, which may be configured to connect a tube 2640, which may be bifurcated at connection 2630 into a suction tube 2610 and an additional working channel 2620. In some embodiments, when required, additional working channel 2620 may be used for passing therethrough various medical instruments configured to perform different medical procedures in the gastrointestinal tract. Those medical instruments may be inserted through opening 2622 into working channel 2620, and then into tube 2640 to reach the lumen of the gastrointestinal tract. In other embodiments, after the clot trimmer tip 2406 performed its operation and the trimmed blood clot debris should be sucked outside of the lumen, suction may be applied via suction tube 2610, and the blood clot debris may be sucked into tube 2640. Then through tube 2610, the debris is transferred (sucked) to a wall mounted suction, which suction tube 2610 is connected to via connector 2611. Tube 2610 may be relatively long to enable the physician to receive assistance from other staff members of the medical team to control the suction trumpet valve 2680.

In some embodiments, a suction trumpet valve 2680 may be connected to suction tube 2610. Suction trumpet valve 2680 may be manually opened or closed by the operator of the blood clot trimmer device, e.g., the gastroenterologist or other physician, as needed. When suction is operated, there is continuous suction within tube 2610, however, knob 2680 may be turned to a closed state, which means that suction would not be applied through tube 2640. Controlling operation of suction during procedure, would ease operation of the blood clot trimmer device by the operator, since the operator does not necessarily want or need continuous suction during exploration of the lumen for other blood clots, etc. However, when the operator does want the application of suction via tube 2640, e.g., such to extract blood clot debris, the operator would turn the knob to its open state, thereby allowing suction pressure to be applied via tube 2640.

As illustrated in FIG. 26B, the blood clot trimmer device with the two-part handle 2400 is inserted through an existing working channel 2660 of the GI device 2600. In addition, there is an external suction channel which is connected to the GI device 2600, and which includes an external cap 2670, tube 2640, external connection 2612, tube 2610 and suction trumpet valve 2680. Distal cap 2670 is attached at the distal tip of the GI device 2600 and connects between the external suction channel and the GI device 2600. External channel tube 2640 is attached to the distal cap 2670 and runs along scope 2602, which includes the imaging device and the working channel of the GI device 2600, until external channel tube 2640 reaches connection 2630, where it is bifurcated to suction tube 2610 and additional working channel 2620. In some embodiments, external connection 2612 is attached to scope 2602 and holds suction tube 2610 and suction trumpet valve 2680, as well as external working channel 2620.

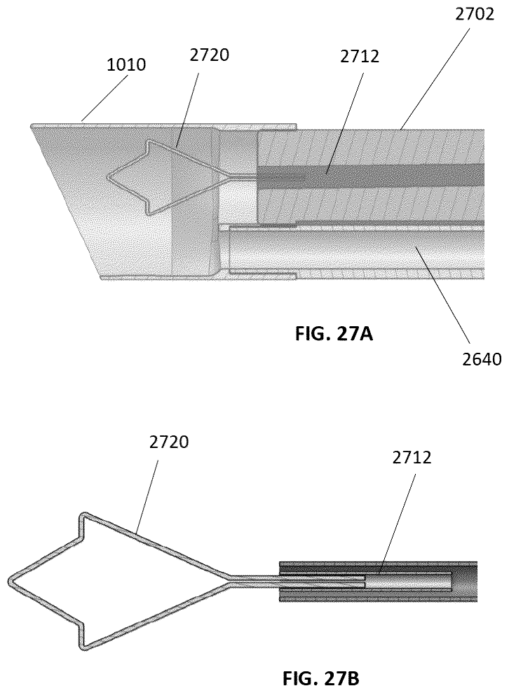

Reference is made to FIGS. 27A-27B, which are schematic illustrations of cross-sections of a blood clot trimmer device tip when the blood clot trimmer tip is outside the sheath but inside the distal cap, and of the clot trimmer tip alone, respectively, according to embodiments of the present disclosure. In some embodiments, the tip of the blood clot trimmer device may be in different shapes. In this embodiment, tip 2720 may have the shape of a ‘duck-foot’. Guide wire 2712 passes through sheath 2702, and in case guide wire 2712 is pushed outside of sheath 2702, tip 2720 expands outside of sheath 2702 to have the form of a ‘duck-foot’ shape. tip 2720 may be rotated by the motor of the blood clot trimmer device, e.g., motor 2510, when the blood clot trimmer comprises a two-part handle 2400, such to trim blood clots within the GI tract. When the user wishes to retract tip 2720 into sheath 2702, the user pulls guide wire 2712 towards the proximal end of the blood clot trimmer device, and the ‘duck-foot’ shaped tip 2720 is forced to fold back into sheath 2702, similarly to tip 3020, described hereinabove with respect to FIGS. 13A-13B.

According to some embodiments, the ‘duck-foot’ shaped trimmer is a six-sided, whereas the diamond shaped tip is a four-sided trimmer. This ‘duck-foot’ six-sided trimmer tip allows for more cutting surface to break up blood clots, compared to the diamond shaped trimmer tip. In addition, the ‘duck-foot’ shaped trimmer tip can be made shorter compared to the diamond shaped trimmer tip, which allows to shorten the length of the distal cap 1010, and thereby reduce ‘tunnel vision’ during operation of the blood clot trimmer, specifically during operation of the clot trimmer tip.

It should be appreciated that the above-described methods and apparatus may be varied in many ways, including omitting or adding elements or steps, changing the order of steps and the type of devices used. It should be appreciated that different features may be combined in different ways. In particular, not all the features shown above in a particular embodiment are necessary in every embodiment of the disclosure. Further combinations of the above features are also considered to be within the scope of some embodiments of the disclosure.

It will be appreciated by persons skilled in the art that the present disclosure is not limited to what has been particularly shown and described hereinabove. Rather the scope of the present disclosure is defined only by the claims, which follow.

What is claimed is:

1. A blood clot trimmer device configured to pass through a work channel that is releasably attachable to an existing endoscope or through a working channel of an existing endoscope, the blood clot trimmer device being configured to trim blood clots and remove trimmed blood from a site internal to a patient body, said blood clot trimmer device comprising:
a flexible guide wire with a distal end and a proximal end;
a sheath configured to enable free passage of the guide wire therethrough, said sheath including a distal end and a proximal end;
a flexible tip connected to the distal end of the guide wire, said tip configured to rotate and thereby trim blood clots;
a tip controller connected to the proximal end of the guide wire and to the proximal end of the sheath, the tip controller configured to control a degree of extraction or retraction of the tip with respect to the distal end of the sheath;
wherein said releasably attachable work channel comprises an endoscopic cap configured to releasably attach onto a distal end of an existing endoscope, the tip of the blood clot trimmer device configured to rotate within the endoscopic cap, and wherein the releasably attachable work channel is configured to operate as a suction channel.

2. The blood clot trimmer device according to claim 1, wherein the tip has a diamond shape.

3. The blood clot trimmer device according to claim 1, wherein the tip has a ‘duck-foot’ shape.

4. The blood clot trimmer device according to claim 1, wherein the tip is made of nitinol.

5. The blood clot trimmer device according to claim 1, wherein the tip controller comprises scale marks to indicate options for changing location of the tip with respect to the sheath.

6. The blood clot trimmer device according to claim 5, wherein the tip controller comprises a tip location lock that is configured to move along the scale marks to determine the degree of extraction or retraction of the tip with respect to the distal end of the sheath, and to lock the determined position of the tip with respect to the distal end of the sheath.

7. The blood clot trimmer device according to claim 1, wherein the blood clot trimmer device comprises a motor connected to the guide wire and a battery to provide power to the motor.

8. The blood clot trimmer device according to claim 7, wherein movement of the motor causes movement of the guide wire and the tip connected to the guide wire with respect to the sheath, thereby determining degree or extraction or retraction of the tip with respect to the distal end of the sheath, further wherein operation of the motor is configured to rotate the tip.

9. The blood clot trimmer device according to claim 1, wherein the blood clot trimmer device is manually operated via user rotation of a threaded handle rotating along a threaded rod.

10. The blood clot trimmer device according to claim 1, wherein diameter of the releasably attachable work channel is between 3.7 mm to 4 mm.

11. The blood clot trimmer device according to claim 1, wherein the blood clot trimmer device is configured to trim blood clots that the releasably attachable work channel is configured to remove via suction, to avoid aspiration of the trimmed blood clots.

12. The blood clot trimmer device according to claim 1, wherein suction via the releasably attachable work channel is configured to operate simultaneously with operation of the blood clot trimmer device to thereby remove trimmed blood clots from the site during operation of the blood clot trimmer device.

13. The blood clot trimmer device according to claim 1, wherein releasably attachable work channel comprises ring shaped connectors to connect between the releasably attachable work channel and the existing endoscope, said ring shaped connectors configured to enable passage of the existing endoscope therethrough.

14. The blood clot trimmer device according to claim 1, further comprising a two-part handle comprising a reusable unit and a disposable unit, wherein the reusable unit comprises the motor and a power source, and wherein the disposable unit comprises the flexible guide wire, the sheath, the flexible tip and the tip controller.

15. The blood clot trimmer device according to claim 14, wherein the reusable unit comprises a protruding shaft, and the disposable unit comprises a corresponding groove configured to house the protruding shaft, wherein when the motor rotates, the shaft rotates, which in turn rotates the walls of the groove, which are connected to the guide wire, which is forced to rotate thereby forcing the flexible tip to rotate and trim blood clots.

16. A method for operating a blood clot trimmer device that is configured to pass through a work channel that is releasably attachable to an existing endoscope or through a working channel of an existing endo scope, the method comprising:
providing a blood clot trimmer device comprising:
a flexible guide wire with a distal end and a proximal end;
a sheath configured to enable free passage of the guide wire therethrough, said sheath including a proximal end and a distal end;
a flexible tip connected to the distal end of the guide wire, said tip configured to rotate and thereby trim blood clots;
a tip controller connected to the proximal end of the guide wire and a proximal end of the sheath, the tip controller configured to control a degree of extraction or retraction of the tip with respect to the sheath;
wherein the releasably attachable work channel comprises an endoscopic cap configured to releasably attach onto a distal end of an existing endoscope;
operating suction via the releasably attachable work channel to suck a blood clot from a site internal to a patient body into the endoscopic cap;
operating rotation of the tip of the blood clot trimer device once a blood clot is sucked into the endoscopic cap, thereby operating a trimming process of the sucked blood clot and breaking the sucked blood clot into pieces;
continuously operating suction by the releasably attachable work/suction channel to remove the pieces of the trimmed blood clot from the site, via the releasably attachable work channel, during the trimming process.

17. The method according to claim 16, further comprising moving a tip location lock along scale marks on the tip controller, thereby determining the degree of extraction or retraction of the tip with respect to the distal end of the sheath.

18. The method according to claim 17, further comprising locking the determined position of the tip with respect to the distal end of the sheath to prevent further movement of the tip with respect to the sheath.

19. The method according to claim 17, wherein the blood clot trimmer device comprises a motor, wherein the method further comprises causing movement of the guide wire and the tip connected to the guide wire with respect to the sheath by moving the motor, thereby determining degree or extraction or retraction of the tip with respect to the distal end of the sheath, and operating the motor for rotating the tip.
